(19) United States
(12) Patent Application Publication
(10) Pub. No.: US 2008/0194456 A1
(43) Pub. Date: Aug. 14, 2008

(54) Compositions And Methods Related To An Intestinal Inflammation And Uses Therefor

(75) Inventor: Daniel Podolsky, Wellesley, MA (US)

Correspondence Address: EWARDS ANGELL PALMER & DODGE LLP;(Client: MGH, Partners), P.O. BOX 55874, BOSTON, MA, 02205, US

(73) Assignee: The General Hospital Corporation, Charlestown, MA (US)

(21) Appl. No.: 10/592,252

(22) PCT Filed: Apr. 11, 2005
(86) PCT No.: PCT/US05/12422

§ 371 Date: Sep. 13, 2007

Related U.S. Application Data
(60) Provisional application No. 60/561,232, filed on Apr. 9, 2004.

(51) Int. Cl. A61K 38/00; C12Q 1/68; A01N 37/18; A61P 1/00

(52) U.S. Cl. 514/2; 435/6

(57) ABSTRACT

Described herein are screening methods to identify therapeutic compositions for the treatment of an intestinal inflammation, inflammatory bowel disease or pathogen infection, as well as therapeutic methods and compositions useful for ameliorating an intestinal inflammation, inflammatory bowel disease or pathogen infection.

### CROSS-REFERENCE TO RELATED APPLICATIONS

This application claims priority to U.S. provisional application Ser. No. 60/561,232, which was filed on Apr. 7, 2005 at Attorney Docket Number 910000-3072, the contents of which are incorporated herein by reference.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims, or in the text itself; and, each of these documents or references (“herein-cited references”), as well as each document or reference cited in each of the herein-cited references (including any manufacturer's specifications, instructions, etc.), is hereby expressly incorporated herein by reference.

### BACKGROUND OF THE INVENTION

It has long been recognized that a variety of microbial agents that are not normally associated with disease may produce “opportunistic” infections in a susceptible host, when the hosts normal defenses are disrupted. In a susceptible host, large numbers of bacterial species that are not normally pathogenic are sometimes found adhering to an intestinal mucosal surface where they manifest low-level epithelial invasion, thus blurring the distinction between pathogens and normal luminal flora. One of these bacterial species is the adhesive invasive E. coli (AIEC). The potential relevance to disease of such bacteria is suggested by the significantly greater frequency of these AIEC in association with Crohn's Disease, which is an inflammatory bowel disease. Inflammatory bowel diseases (IBD) are characterized by chronic inflammation of the intestine.

The pathogenesis of IBD is complex and appears to consist of three interacting elements: genetic susceptibility factors, priming by the enteric microflora, and immune-mediated tissue injury. Although the etiology of IBD remains unclear, a role for microbial agents in the initiation of IBD has been suspected since this disorder was first recognized. Recent studies have shown that there are increased numbers of mucosal adherent and intraepithelial bacteria in patients with IBD, but not in normal control patients, suggesting that IBD may be associated with a functional alteration in the role of intraepithelial cells as the “front-line” of defense against bacteria.

IBD have a devastating effect on quality of life, and are often associated with serious complications, such as stenoses, abscesses, and fistulae that often require repeated surgeries and bowel resections. Standard therapies for IBD focus on controlling disease symptoms without modifying the long-term course of the illness. These therapies often include the long-term use of glucocorticosteroids, which are associated with serious and sometimes irreversible side effects. As current therapies offer limited effectiveness in treating IBD, a need exists for new therapeutic agents and methods for identifying such agents.

### SUMMARY OF THE INVENTION

As will be described in more detail below, the invention provides screening methods to identify therapeutic compositions for the treatment of an intestinal inflammation, inflammatory bowel disease or pathogen infection, as well as therapeutic methods and compositions useful for ameliorating an intestinal inflammation, inflammatory bowel disease or pathogen infection.

In one embodiment, the invention provides a method for identifying a compound that decreases an intestinal inflammation, the method comprising the steps of: (a) contacting a cell expressing a GRIM-19 nucleic acid molecule with a candidate compound; and (b) detecting an increase in GRIM-19 expression in the contacted cell relative to expression of a reference nucleic acid molecule, wherein an increase in GRIM-19 expression identifies the candidate compound as useful for decreasing an intestinal inflammation, thereby identifying a compound that decreases an intestinal inflammation. In specific embodiments, the method can identify a compound that increases GRIM-19 transcription or translation. Expression can be detected, for example, using a polymerase chain reaction (e.g., real time PCR) or a reverse transcription polymerase chain reaction.

In another embodiment, the invention provides a method for identifying a compound that decreases an intestinal inflammation, the method comprising the steps of: (a) contacting a cell expressing a GRIM-19 polypeptide with a candidate compound; and (b) detecting an increase in the amount of GRIM-19 polypeptide in the cell contacted with the candidate compound relative to an amount of a reference polypeptide, wherein an increase in the amount of GRIM-19 polypeptide identifies the candidate compound as useful for decreasing an intestinal inflammation, thereby identifying a compound that decreases an intestinal inflammation.

In yet another embodiment, the invention provides a method for identifying a compound that decreases an intestinal inflammation, the method comprising the steps of: (a) contacting a cell expressing a GRIM-19 polypeptide with a candidate compound; and (b) comparing the biological activity of the GRIM-19 polypeptide in the cell contacted with the candidate compound with the biological activity of the GRIM-19 polypeptide in a control cell, wherein an increase in the biological activity of the GRIM-19 polypeptide identifies the candidate compound as useful for decreasing an intestinal inflammation, thereby identifying a compound that decreases an intestinal inflammation. The biological activity can be monitored with an enzymatic assay, such as an enzymatic assay that detects nicotinamide adenine dinucleotide phosphate dehydrogenase activity. The biological activity can also be monitored with an NF-κB activation assay, a bacterial invasion assay or an immunological assay, such as an immunological assay that detects GRIM-19 binding to NOD2.

In yet another embodiment, the invention provides a method of identifying a compound that decreases an intestinal inflammation, the method comprising the steps of: a) contacting a cell comprising a GRIM-19 promoter operably linked to a detectable reporter gene with a candidate compound; and b) comparing the amount of reporter gene expression in the cell contacted with the candidate compound with a control cell not contacted with the candidate compound, wherein an increase in the amount of the reporter gene expression identifies the candidate compound as useful for decreasing an intestinal inflammation, thereby identifying a compound that decreases an intestinal inflammation.

Assays of the invention for identification of a compound that decreases an intestinal inflammation can be conducted in a cell in vitro or in vivo. The cell can be, for example, an intestinal epithelial cell. Such assays can include high throughput screening methods.

In yet another embodiment, the invention provides a method for identifying a compound that decreases an intestinal inflammation, the method comprising the steps of: (a) contacting a GRIM-19 polypeptide with a candidate compound; (b) detecting binding of the GRIM-19 polypeptide with the candidate compound; and (c) monitoring the biological activity of the Grim 19 polypeptide, wherein an increase in the biological activity of the GRIM-19 polypeptide is useful for decreasing an intestinal inflammation, thereby identifying a compound that decreases an intestinal inflammation. The binding can be detected in a cell (e.g., intestinal epithelial cell).

Compounds identified according to methods of the invention can also alter a host response to a microbe, such as a bacteria. The candidate compounds can be but are not limited to small molecules, a nucleic acid molecules and polypeptides. Such candidate compounds can be antibiotics that are useful for treating an infection or inflammation that occurs anywhere in the body.

In yet another embodiment, the invention provides an isolated intestinal epithelial cell comprising a recombinant GRIM-19 nucleic acid molecule.

In yet another embodiment, the invention provides a method for diagnosing a subject having, or having a propensity to develop, an intestinal inflammation, the method comprising detecting an alteration in the sequence of a GRIM-19 nucleic acid molecule relative to a wild-type sequence of a GRIM-19 nucleic acid molecule.

In yet another embodiment, the invention provides a method for diagnosing a subject having, or having a propensity to develop, an intestinal inflammation, the method comprising detecting an alteration in the expression of a GRIM-19 nucleic acid molecule or polypeptide relative to the wild-type level of expression of the GRIM-19 nucleic acid molecule or polypeptide.

In yet another embodiment, the invention provides a method for diagnosing a subject having, or having a propensity to develop, an intestinal inflammation, the method comprises detecting an alteration in the biological activity of a GRIM-19 polypeptide relative to the wild-type level of activity.

In yet another embodiment, the invention provides a method for ameliorating an intestinal inflammation in a subject, the method comprising contacting the subject with one or more compounds that increase GRIM-19 nucleic acid or polypeptide expression, thereby ameliorating the intestinal inflammation in the subject.

In yet another embodiment, the invention provides a method for ameliorating an intestinal inflammation in a subject, the method comprising contacting the subject with one or more compounds that increase GRIM-19 activity, thereby ameliorating the intestinal inflammation in the subject. One of the compounds can be an interferon, a retinoic acid, a substrate or activator of Grim 19 oxidoreductase (e.g., an enzyme cofactor). In specific embodiments, the compounds are a combination of an interferon and retinoic acid.

The intestinal inflammation can be an inflammatory bowel disease, such as Crohn's disease or ulcerative colitis.

In yet another embodiment, the invention provides a method for reducing a pathogen infection in a subject, the method comprising contacting the subject with one or more compounds that increase GRIM-19 nucleic acid or polypeptide expression, thereby reducing the pathogen infection in the subject.

In yet another embodiment, the invention provides a method for reducing a pathogen infection in a subject, the method comprising contacting the subject with a one or more compounds that increase GRIM-19 activity, thereby reducing a pathogen infection in a subject.

In yet another embodiment, the invention provides a method for inactivating a pathogen in an epithelial cell, the method comprising providing the cell with a GRIM-19 nucleic acid molecule or polypeptide, or an activator thereof.

The pathogen can be a bacteria, such as E. coli or S. typhimuriam. Preferably, the therapeutic methods of the invention inhibit the growth or survival of the bacteria.

In yet another embodiment, the invention provides a pharmaceutical composition comprising an effective amount of a GRIM-19 polypeptide, or fragment thereof, in a pharmacologically acceptable excipient.

In yet another embodiment, the invention provides a pharmaceutical composition comprising an effective amount of a GRIM-19 nucleic acid molecule, or fragment thereof, in a pharmacologically acceptable excipient.

In yet another embodiment, the invention provides a biocide comprising an effective amount of a GRIM-19 polypeptide or nucleic acid molecule, or fragment thereof, in a biocide excipient.

In yet another embodiment, the invention provides a method of inhibiting microbial growth in a cell, the method comprising providing an effective amount of a biocide comprising a GRIM-19 polypeptide or a nucleic acid molecule or fragment thereof to a cell containing the microbe.

In yet another embodiment, the invention provides a diagnostic kit for detecting a GRIM-19 polypeptide comprising an agent capable of detecting a GRIM-19 polypeptide in a biological sample.

In yet another embodiment, the invention provides a diagnostic kit of claim 45, wherein the agent is an antibody that specifically binds to GRIM-19. The kit can further comprises a reference standard.

In yet another embodiment, the invention provides a diagnostic kit for detecting a GRIM-19 nucleic acid molecule, the kit comprising an oligonucleotide capable of hybridizing with a GRIM-19 nucleic acid molecule. The kit can further comprise at least two primers capable of binding to and amplifying a GRIM-19 nucleic acid molecule.

In yet another embodiment, the invention provides a method for identifying a compound that decreases an intestinal inflammation, the method comprising the steps of: (a) contacting a cell expressing a NOD2 nucleic acid molecule with a candidate compound; and (b) detecting an increase in NOD2 expression in the contacted cell relative to expression of a reference nucleic acid molecule, wherein an increase in NOD2 expression identifies the candidate compound as a candidate compound useful for decreasing an intestinal inflammation, thereby identifying a compound that decreases an intestinal inflammation. In specific embodiments, the that increases translation of an mRNA transcribed from the NOD2 nucleic acid molecule.

In yet another embodiment, the invention provides a method for identifying a compound that decreases an intestinal inflammation, the method comprising the steps of: (a) contacting a cell expressing a NOD2 promoter operably linked to a detectable reporter with a candidate compound; and (b) detecting an increase in reporter expression in the contacted cell relative to a reference, wherein an increase in reporter expression identifies the candidate compound as useful for decreasing an intestinal inflammation, thereby identifying a compound that decreases an intestinal inflammation.

In yet another embodiment, the invention provides a method for identifying a compound that decreases an intestinal inflammation, the method comprising the steps of: (a) contacting a cell expressing a NOD2 polypeptide with a candidate compound; and (b) detecting an increase in the amount of NOD2 polypeptide in the cell contacted with the candidate compound relative to an amount of a reference polypeptide, wherein an increase in the amount of NOD2 polypeptide identifies the candidate compound as useful for decreasing an intestinal inflammation, thereby identifying a compound that decreases an intestinal inflammation.

In yet another embodiment, the invention provides a method for identifying a compound that decreases an intestinal inflammation, the method comprising the steps of: (a) contacting a cell expressing a NOD2 polypeptide with a candidate compound; and (b) comparing the biological activity of the NOD2 polypeptide in the cell contacted with the candidate compound with the biological activity in a control cell, wherein an alteration in the biological activity of the NOD2 polypeptide identifies the candidate compound as useful for decreasing an intestinal inflammation, thereby identifying a compound that decreases an intestinal inflammation.

In yet another embodiment, the invention provides a method for identifying a compound that decreases an intestinal inflammation, the method comprising the steps of: (a) contacting a cell expressing a NOD2 polypeptide with a candidate compound; and (b) detecting binding of the candidate compound to NOD2, wherein a compound that binds a NOD2 polypeptide is useful for decreasing an intestinal inflammation, thereby identifying a compound that decreases an intestinal inflammation.

NOD2-based assays of the invention for identification of a compound that decreases an intestinal inflammation can be conducted in a cell in vitro or in vivo. The cell can be, for example, an intestinal epithelial cell. Cells expressing NOD2 can further comprises a GRIM-19 polypeptide. Such assays can include high throughput screening methods. Compounds identified according to methods of the invention can also alter a host response to a microbe, such as a bacteria. The candidate compounds can be but are not limited to small molecules, a nucleic acid molecules and polypeptides. Such candidate compounds can be antibiotics that are useful for treating an infection or inflammation that occurs anywhere in the body.

In yet another embodiment, the invention provides an intestinal epithelial cell comprising a recombinant NOD2 nucleic acid molecule.

In yet another embodiment, the invention provides a substantially pure antibody that specifically binds a NOD2 polypeptide.

Other features and advantages of the invention will be apparent from the detailed description, and from the claims.

### BRIEF DESCRIPTION OF THE DRAWINGS

FIGS. 1A-1D are photographs of agarose gels containing RT-PCR products. FIG. 1A shows CARD4/NOD1 mRNA expression in the indicated intestinal epithelial cell (IEC) lines relative to GAPDH expression, as shown in FIG. 1B. FIG. 1C shows CARD15/NOD2 expression in IEC lines relative to GAPDH expression, which is shown in FIG. 1D.

FIGS. 2A-2D show CARD15/NOD2 and CARD4/NOD1 mRNA expression in IEC lines. FIGS. 2A-2C are photomicrographs showing a whole crypt, an intestinal epithelial cell, and a lymphocyte. FIG. 2D is a set of four photographs of agarose gels containing RT-PCR products for NOD2, CD45, GPDH, and an RT-PCR product using total RNA from a single Jurkat cell and THP-1 cell.

FIGS. 3A, B, C, D, E, and F are photographs showing Northern blots. FIG. 3A shows a Northern blot analysis of Nod1 mRNA expression in SW480 cells following addition of 10 ng/ml of IFNγ, TNFα, IL-1β, IL-4, and TGFβ for six hours relative to GAPDH expression, which is shown in FIG. 3B. FIG. 3C shows the concentration dependent effects of IFNγ on Nod1 mRNA expression in SW480 cells relative to GAPDH expression, which is shown in FIG. 3D. Cells were treated with IFNγ for six hours. FIG. 3E shows the time dependent effects of IFNγ on Nod1 mRNA expression in SW480 cells relative to GAPDH expression, which is shown in FIG. 3F. Cells were treated with 100 ng/ml IFNγ.

FIGS. 4A and 4B are photographs of Western blots. FIG. 4A shows a Western blot of CARD4/NOD1 protein in COS7 cells transiently transfected with pCl CARD4-HA plasmid. Anti-HA monoclonal antibody and biotinylated anti-CARD4/NOD1 antiserum (HM3847) detected NOD1 protein in COS7 cells transiently transfected with pCl CARD4-HA. FIG. 4B shows a Western blot of protein obtained from SW480 cells cultured with 100 ng/ml of IFNγ for six, twelve, twenty-four, and forty-eight hours were immunoprecipitated with affinity purified anti-Nod 1 antiserum (HM3851) and then immunoblotted with biotinylated anti-CARD4/NOD1 antiserum (HM3847). Lysates from COS7 cells transiently transfected with pCl CARD4-HA were used as positive control.

FIGS. 5A-5D are panels showing that IFNγ activated the human NOD1 promoter. FIG. 5A is a schematic diagram depicting the CARD4/NOD1 gene. Three IRF-1 binding motifs (IRF-1A, IRF-1B, and IRF-1C) are indicated as black boxes (▪). FIG. 5B is a schematic diagram depicting pGL Nod 1-luciferase deletion mutants. FIGS. 5C and 5D are graphs showing luciferase activity in SW480 cells were transiently transfected with 500 ng/well of the indicated expression plasmid. Ten hours after transfection, cells were cultured for a further sixteen hours with 100 ng/ml of IFNγ and then luciferase activity was measured. Black bars indicate the presence of IFNγ. White bars indicate the control conditions.

FIG. 6 is a photograph of electrophoretic mobility shift assay that shows the interaction of IRF-1 with the IRF-1 binding motifs of the human Nod1 promoter. The competition assay was performed with a 100-fold excess of unlabeled oligonucleotides. The supershift assays were done by the addition of 1 μg of anti-IRF-1 antibody.

FIG. 7 is a graph showing that IRF-1 activates CARD4/NOD1 transcription in SW480 cells. Transcription was assayed by measuring luciferase activity in SW480 cells co-transfected with 500 ng/well of pGLb (control), pGL-2128, or pGLΔ-837-546 and the indicated amount of the IRF-1 expression plasmid.

FIGS. 8A-8D are photographs of Northern blots showing the effect of cytokines on Nod2 mRNA expression in SW480 cells. FIG. 8A is a Northern blot analysis of Nod2 mRNA expression in SW480 cells treated with 10 ng/ml of IFNγ, TNFα, IL-1β, IL-4 and TGFβ for six hours. As a positive control for CARD15/NOD2, human PBMC was used. FIG. 8B shows the concentration dependent effects of TNFα on Nod2 expression. FIG. 8C shows the time dependent effects of TNFα on Nod2 expression. In each of FIGS. 8A-8C, expression of Nod2 is shown relative to GAPDH expression. FIG. 8D shows the effect of cycloheximide on expression of NOD2 mRNA in SW480 cells.

FIGS. 9A-9D depict NOD2 protein and its expression in SW480 cells. FIG. 9A is a schematic diagram showing the location of polypeptide sequences of NOD2 for immunization. FIGS. 9B, 9C, and 9D are photographs of Western blots. FIG. 9B shows NOD2 protein expression in COS7 cells transiently transfected with pCMV FLAG-NOD2. Untransfected COS7 cells were used as a negative control. FIG. 9C shows the results of an immunoprecipitation and Western blot of NOD2. Transiently transfected COS7 cells were immunoprecipitated with affinity purified (a.p.) anti-NOD2 antiserum (HM2559 a.p.), then immunoblotted with affinity purified biotinylated anti-NOD2 antiserum (b-HM2563 a.p.). FIG. 9D shows NOD2 protein expression in SW480 cells. As a positive control for NOD2 protein, lysates of COS7 cells transfected with pCMV FLAG-NOD2 was used.

FIG. 10 is a set of three photographs of Western Blots showing that NOD2/CARD15 interacts with GRIM-19 tagged with an Xpress protein tag following bacterial invasion. Xp=xpress protein tag.

FIGS. 11A-11D are panels showing the effect of wild type NOD2 and mutant NOD2 (3020insC) on cell resistance to S. typhimurium infection. FIG. 11A is a photograph of an agarose gel with separated RT-PCR products. The RT-PCR products correspond to N-terminal and C-terminal sequences of CARD15/NOD2 in Caco2, MOCK, NOD2-Caco2, and 3020insC-Caco2 cells.

FIG. 11B is a photograph of a Western blot showing CARD15/NOD2 protein expression by immunoblotting with anti-CARD15/NOD2 sera (immunoprecipitation:HM2563 a.p., immunoblot:b-HM2563 a.p.) in Caco2, MOCK, NOD2-Caco2, and 3020insC-Caco2 cells. As positive controls for NOD2 and 3020insC protein, lysates of COS7 cells transfected with pCMV FLAG-NOD2 and pCMV FLAG-3020insC were used, respectively. FIG. 11C is a photograph of a Western blot showing CARD15/NOD2 protein expression was shown both in NOD2-Caco2 cells and SW480 cells treated with 100 ng/ml of TNFα for forty-eight hours. One mg of lysate was immunoprecipitated with HM2559 a.p. and immunoblotted with b-HM2563. As a positive control for NOD2/CARD15 protein, the lysate of COS7 cells transfected with pCMV FLAG-NOD2 was used. FIG. 11D is a graph showing the results of a gentamicin protection assay of S. typhimurium in Caco2, MOCK, NOD2-Caco2, and 3020insC cells. Experiments were performed in quadruplicate. The data are presented as the average % CFU (the percentage of original inoculation, mean ±SD) with untransfected Caco2 values set as 100%. Results were confirmed in three independent experiments.

FIGS. 12A-12C are photomicrographs showing the cellular localization of wild type or mutant Nod2 tagged with GFP in transiently transfected cells. FIG. 12A shows the cellular localization of mutant NOD2. FIG. 12B shows the cellular localization of wild-type NOD2; FIG. 12C shows the cellular localization of wild-type NOD2 following invasion with red-tagged salmonella.

FIGS. 13A-13D depict the association between CARD15/NOD2 and GRIM-19 in mammalian cells. FIG. 13A is a schematic diagram that depicts the full length and CARD15-less NOD2 construct constructs used as bait for yeast two-hybrid screening. CARD: caspase recruitment domain; NBD: nucleotide binding domain; LRR: leucine-rich repeat (LRR) region FIG. 13B is a set of six panels, each of which shows a photograph of an immunoblot. For each immunoblot, COS7 cells were transfected with Flag-tagged NOD2 and/or Xpress-tagged GRIM-19, then the cell lysates were immunoprecipitated with anti-Flag antibody (IP Flag) (Upper left panel) or with anti-Xpress antibody (IP Xpress) (Upper right panel). The precipitates were fractionated through 4-12% or 4-20% Tris-Glycine SDS-PAGE, and blotted with anti-Xpress (Left and right upper panels) or anti-Flag (Left and right middle panels) monoclonal antibodies. Total cell lysate (TCL) were subjected to Western blot analysis with anti-Xpress antibody (Left bottom panel) or anti-Flag antibody (Right bottom panel) to detect the expression of GRIM-19 or NOD2 in transfected COS7 cells. FIG. 13C is a set of three panels, each of which shows a photograph of an immunoblot. For each immunoblot, HT29 cells were transfected with Xpress-tagged GRIM-19. The cell lysates were immunoprecipitated with anti-GRIM-19 antibody (IP GRIM-19) then subjected to Western blot analysis using rabbit anti serum against human NOD2 (Upper panel) or human GRIM-19 (Middle panel). Total cell lysates subjected to Western blot analysis using rabbit anti serum against human NOD2 is shown in the bottom panel. FIG. 13D is a set of four panels, each of which shows a photograph of an immunoblot. For each immunoblot COS7 cells were transfected with a control vector, Xpress-tagged GRIM-19 (Xp-GRIM-1 g), CARD4/NOD 1-HA tagged (NOD1-HA), or both (Xp-GRIM-19+NOD1-HA). After immunoprecipitation with anti-Xpress (IP Xpress) (Upper two panels) or anti-HA (IP HA) (Bottom two panels) monoclonal antibodies, immunoprecipitates were subjected to Western blot analysis using anti-Xpress (WB Xpress) or anti-HA monoclonal antibodies.

FIG. 14 is a set of six photomicrographs showing that GFP-NOD2 and Xpress-GRIM-19 colocalize in mammalian Caco-2 and COS7 cells co-transfected with GFP-tagged NOD2 and Xpress-tagged GRIM-19. GRIM-19 was detected by confocal microscopy using monoclonal anti-Xpress as the primary antibody and Texas Red-conjugated anti-mouse IgG as the secondary antibody.

FIGS. 15A and 15B show grim-19 expression in inflammatory bowel tissues (FIG. 15A) and different cell lines (FIG. 15B). FIG. 15A is a graph showing grim-19 mRNA expression level relative to a GAPDH mRNA internal standard. RT-PCR was used to determine grim-19 mRNA content in biopsies isolated from involved and non-involved areas of colonic mucosa from four patients diagnosed with Crohn's disease (CD), five patients diagnosed with ulcerative colitis (UC), and three normal control patients without inflammatory bowl disease. * p<0.05 FIG. 15B is a photograph of an agarose gel containing RT-PCR products (194 bp) showing that grim-19 mRNA is present in the following cell lines: IEC, THP-1, Jurkat, COS7 and HEK293 cell lines. GAPDH (440 base pairs) was used as an internal control. The identity of all fragments was confirmed by sequencing.

FIG. 16 is a graph showing grim-19 mRNA expression level relative to a GAPDH mRNA internal standard in Caco-2 cell monolayers that were previously infected with S. typhimurium or with a non-pathogenic E. coli as compared to uninfected control cells.

FIGS. 17A-17D show that GRIM-19 expression protected host cells from cellular damage caused by S. typhimuriam infection. FIG. 17A is a graph that shows the release of adenylate kinase (AK) from damaged cells. Cells were transfected with one of the following vectors: Xpress-tagged GRIM-19, Flag-tagged NOD2 or pcDNA4 control vector. The cells were then infected with S. typhimurium for two hours at a MOI=50. Levels of AK release are normalized to that of untransfected Caco-2 cells, which are assigned a value of one. FIG. 17B is a graph showing the percentage of intracellular S. typhimurium in Caco-2 cells transfected with Xpress-tagged GRIM-19, grim-19 siRNA-1, control grim-19 siRNA, or control vectors (pcDNA4 and pSUPER). Intracellular bacterial infection was quantified after a two hour infection period at a MOI=10 followed by a one hour treatment with gentamicin. Results are shown relative to the percentage of intracellular bacteria present in untransfected Caco-2 cells, which were assigned a value of 100%. Each value is the mean ±SEM of at least five separate experiments. FIG. 17C is a photomicrograph showing confocal analysis of Caco-2 cells transfected with Xpress-GRIM-19 and infected with S. typhimurium. GRIM-19 was detected using anti-Xpress monoclonal antibody and visualized with a Texas Red-conjugated anti-mouse IgG. S. typhimurium was detected using a fluorescein conjugated rabbit antibody against Salmonella. The nucleus of each infected cell is indicated with an “N.” FIG. 17D shows the mean number of bacteria per cell present in fifty Xpress-tagged GRIM-19 transfected cells and fifty untransfected cells. * p<0.05, ** p<0.01

FIG. 18 is a graph that illustrates the invasive ability of S. typhimurium in Caco-2 cells stimulated with a combination of retinoic acid (RA) and interferon-α (IFN-α) to induce endogenous GRIM-19 expression. The percentage of intracellular bacteria was determined as described above. grim-19 mRNA levels were determined by RT-PCR as described above. Controls cells were tranfected with an empty vector (pSUPER). * p<0.05

FIGS. 19A and 19B are graphs showing that GRIM-19 acts downstream of NOD2 and is required from NF-κB activation. FIG. 19A shows NF-κB luciferase reporter activity in HEK293 cells twenty-four hours after transfection with one or more of the following: 1 μg of NOD2 expression plasmid, 10 μg of grim-19 siRNA-1, 10 ng of control grim-19 siRNA, or an empty vector (pSUPER) control. NF-κB activity was determined in the absence or presence of 1 μg of MDP-LD. grim-19 mRNA level was measured by RT-PCR using specific primers. FIG. 19B shows the percentage of intracellular bacteria present in HEK293 cells transfected with 1 ng of NOD2 expression plasmid, 10 ng of grim19 siRNA-1, or with an empty vector control (pSuper) and stimulated with 1 μg of MDP-LD. After a two hour period of infection with S. typhimurium at MOI=10, invasive bacteria were quantified as described above. * p<0.05

### DETAILED DESCRIPTION OF THE INVENTION

### Definitions

By “ameliorate” is meant decrease, suppress, attenuate, diminish, arrest, or stabilize the development or progression of a disease.

By “antibody” is meant any immunoglobulin polypeptide, or fragment thereof, having immunogen binding ability.

By “biocide” is meant any agent that directly kills or attenuates the survival by inhibiting the growth or replication of a microbe.

The terms “comprises,” “comprising,” “containing” and “having” have the meaning ascribed to them in U.S. patent law and can mean “includes,” “including,” and the like; “consisting essentially of” or “consists essentially” likewise has the meaning ascribed in U.S. patent law. Thus, the term is open-ended and allows for the presence of more than that which is recited so long as the basic or novel characteristics of that which is recited is not changed by the presence of more than that which is recited, but excludes prior art embodiments.

By “disease” is meant any condition or disorder that damages or interferes with the normal function of a cell, tissue, or organ. Examples of diseases include bacterial invasion or colonization of a host cell.

By “fragment” is meant a portion of a protein or nucleic acid that is substantially identical to a reference protein or nucleic acid. In some embodiments the portion retains at least 50%, 75%, or 80%, or more preferably 90%, 95%, or even 99% of the biological activity of the reference protein or nucleic acid described herein.

By a “GRIM-19 polypeptide” is meant a protein that is substantially identical to the amino acid sequence of GenBank Accession No. NP_{—}057049, or a fragment thereof, and having at least one GRIM-19 biological activity.

By “GRIM-19 biological activity” is meant NFκB activation, NOD2 binding, NADH dehydrogenase (ubiquinone) activity, oxidoreductase activity, an anti-bacterial activity, or an innate mucosal response.

By a “grim-19 nucleic acid molecule” is meant a nucleic acid molecule that encodes any GRIM-19 polypeptide or fragment thereof. Exemplary grim-19 nucleic acid molecules include NM_{—}015965 (Chidambaram et al., J. Interferon Cytokine Res. 20: 661-665, 2000)

By “intestine” is meant the lower part of the alimentary canal, which extends from the stomach to the anus and is composed of a convoluted upper part (small intestine) and a lower part of greater diameter (large intestine).

By “intestinal epithelial cell” is meant a cell contained within the tissues that cover the lumenal surface of the intestine, including, but not limited to, absorptive cells of the small intestine, columnar epithelial cells of the large intestine, endocrine cells (large and small intestine), and crypt cells (including mucous gland cells, serous gland cells, and stem cells).

By “intestinal inflammation” is meant an inflammatory response that interferes with the normal function of the intestine. Methods of detecting intestinal inflammation are known to the skilled artisan. In one embodiment, gastrointestinal inflammation is assessed using an upper GI series, flexible sigmoidoscopy, colonoscopy, biopsy of an affected intestinal tissue, intestinal x-ray, CT scan or other imaging studies. In one embodiment, intestinal inflammation is detected using the commercially available diagnostic, IBD-CHEK®, which is an ELISA that can be used to identify patients with active inflammatory bowel disease (IBD), which result in elevated levels of fecal lactoferrin.

By “inflammatory bowel disease” is meant a condition of chronic intestinal inflammation. Exemplary inflammatory bowel diseases (IBD) include ulcerative colitis and Crohn's disease.

By “intestinal cell specific promoter” is meant a promoter that directs expression of an operably linked DNA sequence when bound by transcriptional activator proteins, or other regulators of transcription, which are unique to an intestinal cell (e.g., an intestinal epithelial cell, or a specific type of intestinal epithelial cell (e.g., small intestine cell, large intestine cell, glandular cell, or absorptive cell)). In one embodiment, an intestinal cell specific promoter that directs expression in an intestinal epithelial cell includes sucrase, lactase-phlorizin hydrolase, and carbonic anhydrase promoters. Exemplary intestinal cell promoters are described in Boll et al. 1991 Am. J. Hum. Genet. 48:889-902; Brady et al. 1991 Biochem. J. 277:903-5; Drummond et al. 1996 Eur. J. Biochem. 236:670-81; Olsen et al. 1994 FEBS Lett. 342:325-8; Rodolosse et al. 1996 Biochem. J. 315:301-6; Sowden et al. 1993 Differentiation 53:67-74; Traber 1990 Biochem. Biophys. Res. Commun. 173:765-73; Traber et al. 1992 Mol. Cell. Biol. 12:3614-27; Troelsen et al. 1994 FEBS Lett. 342:291-6; Troelsen et al. 1994 FEBS Lett. 342:297-301; and Troelsen et al. 1992 J. Biol. Chem. 267:20407-11.

By “isolated nucleic acid molecule” is meant a nucleic acid (e.g., a DNA) that is free of the genes that, in the naturally occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By “large intestine” is meant the region of the intestine composed of the ascending colon, transverse colon, descending colon, sigmoid colon, and rectum.

By “microbe” is meant a single-celled organism. Microbes include pathogenic organisms, and organisms that are not typically pathogenic.

By “pathogen” is meant any bacteria, viruses, fungi, or protozoans capable of interfering with the normal function of a cell.

Exemplary bacterial pathogens include, but are not limited to, Aerobacter, Aeromonas, Acinetobacter, Agrobacterium, Bacillus, Bacteroides, Bartonella, Bordetella, Brucella, Calymmatobacterium, Campylobacter, Citrobacter, Clostridium, Cornyebacterium, Enterobacter, Escherichia, Francisella, Haemophilus, Hafnia, Helicobacter, Klebsiella, Legionella, Listeria, Morganella, Moraxella, Proteus, Providencia, Pseudomonas, Salmonella, Serratia, Shigella, Staphylococcus, Streptococcus, Treponema, Xanthomonas, Vibrio, and Yersinia.

By “reference” is meant a standard or control condition.

By “NOD2 polypeptide” is meant a protein that is substantially identical to the amino acid sequence of GenBank Accession No. CAC42117, or a fragment thereof, and having at least one NOD2 biological activity.

By “NOD2 nucleic acid molecule” is meant a polynucleotide that encodes a NOD2 polypeptide or fragment thereof. An exemplary NOD2 nucleic acid sequence is provided by GenBank Accession No. AJ303140.

By “NOD2 biological activity” is meant NF-κB activation, an anti-bacterial activity, an innate mucosal response or GRIM-19 binding.

By “promoter” is meant a minimal DNA sequence sufficient to direct transcription.

By “protein” is meant a polypeptide (native or mutant), oligopeptide, peptide, or other amino acid sequence. As used herein, “protein” is not limited to native or full-length proteins, but is meant to encompass protein fragments having a desired activity or other desirable biological characteristic, as well as mutants or derivatives of such proteins or protein fragments that retain a desired activity or other biological characteristic. Mutant proteins encompass proteins having an amino acid sequence that is altered relative to a reference sequence. Such alterations include, but are not limited to, amino acid substitutions (conservative or non-conservative), deletions, or additions (e.g., as in a fusion protein). “Protein” and “polypeptide” are used interchangeably herein without intending to limit the scope of either term.

A “subject” is a mammal. Mammals include, but are not limited to, humans, farm animals, sport animals, and pets.

By “substantially identical” is meant a polypeptide or nucleic acid molecule exhibiting at least 85% identity to a reference amino acid sequence or nucleic acid sequence. Preferably, such a sequence is at least 85%, 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “transformation” is meant a transient (i.e., episomal or otherwise non-inheritable) or permanent (i.e., stable or inheritable) genetic change induced in a cell following incorporation of new DNA (i.e., DNA exogenous to the cell).

By “transformed cell” is meant a cell into which (or into an ancestor of which) has been introduced, by means of recombinant nucleic acid techniques, a nucleic acid molecule, i.e., a sequence of codons formed of nucleic acids (e.g., DNA or RNA) encoding a protein of interest. The introduced nucleic acid sequence may be present as an extrachromosomal or chromosomal element.

By “polypeptide” is meant any chain of amino acids, regardless of length or post-translational modification.

### METHODS OF THE INVENTION

The invention features compositions and methods useful for the treatment of an intestinal inflammation, inflammatory bowel disease or pathogen infection, as well as screening methods for the identification of therapeutic compounds useful for the treatment of an intestinal inflammation, inflammatory bowel disease or pathogen infection. These methods and compositions are based, in part, on the discoveries that GRIM-19 regulates intestinal epithelial cell responses to microbes via its interaction with NOD2, a protein that acts as a bacterial sensor, and that GRIM-19 and NOD2 are expressed in intestinal epithelials cells (IEC) where they function as key components of the innate mucosal response to pathogens. Accordingly, the invention provides the following methods and materials.

### Screening Assays

The expression of GRIM-19, which is a NOD2 binding partner and a key component of the innate mucosal response to pathogens, is reduced in inflammatory bowel disease. Based in part on this discovery, compositions of the invention are useful for the high-throughput screening of candidate compounds to identify those that increase the expression of GRIM-19. In one embodiment, the effects of known candidate compounds on the expression of GRIM-19 are assayed. Tissues or cells treated with a candidate compound are compared to untreated control samples to identify therapeutic agents that increase the expression of a GRIM-19 polypeptide or nucleic acid molecule. Any number of methods are available for carrying out screening assays to identify new candidate compounds that promote the expression of a GRIM-19 polypeptide or nucleic acid molecule.

In one working example, candidate compounds are added at varying concentrations to the culture medium of cultured cells expressing one of the nucleic acid sequences of the invention. Gene expression is then measured, for example, by microarray analysis, Northern blot analysis (Ausubel et al., supra), reverse transcriptase PCR, or quantitative real-time PCR using any appropriate fragment prepared from the nucleic acid molecule as a hybridization probe. The level of gene expression in the presence of the candidate compound is compared to the level measured in a control culture medium lacking the candidate molecule. A compound that promotes an increase in the expression of a GRIM-19 nucleic acid molecule, or a functional equivalent thereof, is considered useful in the invention; such a candidate compound may be used, for example, as a therapeutic to treat an intestinal inflammation, inflammatory bowel disease or pathogen infection in a subject.

In another working example, the effect of a candidate compound is measured at the level of polypeptide production using the same general approach and standard immunological techniques, such as Western blotting or immunoprecipitation with an antibody specific for a GRIM-19 polypeptide or for a GRIM-19/NOD2 complex. For example, immunoassays may be used to detect or monitor the expression of at least one of the polypeptides of the invention in an organism. Polyclonal or monoclonal antibodies that are capable of binding to a polypeptide of the invention may be used in any standard immunoassay format (e.g., ELISA, Western blot, or RIA assay) to measure the level of the polypeptide. In some embodiments, a compound that promotes an increase in the expression or biological activity of the polypeptide is considered particularly useful. Again, such a candidate compound may be used, for example, as a therapeutic to delay, ameliorate, or treat an intestinal inflammation, inflammatory bowel disease or pathogen infection, or their symptoms in a subject.

In yet another working example, candidate compounds are screened for those that specifically bind to a GRIM-19 polypeptide or a GRIM-19NOD2 polypeptide complex. The efficacy of such a candidate compound is dependent upon its ability to interact with such a polypeptide or complex, or with functional equivalents thereof. Such an interaction can be readily assayed using any number of standard binding techniques and functional assays (e.g., those described in Ausubel et al., supra). In one embodiment, a candidate compound may be tested in vitro for its ability to specifically bind a polypeptide of the invention.

In one particular working example, a candidate compound that binds to a GRIM-19 polypeptide is identified using a chromatography-based technique. For example, a recombinant polypeptide of the invention may be purified by standard techniques from cells engineered to express the polypeptide (e.g., those described above) and may be immobilized on a column. A solution of candidate compounds is then passed through the column, and a compound specific for the GRIM-19 polypeptide is identified on the basis of its ability to bind to the polypeptide and be immobilized on the column. To isolate the compound, the column is washed to remove non-specifically bound molecules, and the compound of interest is then released from the column and collected. Similar methods may be used to isolate a compound bound to a polypeptide microarray.

In another example, the compound, e.g., the substrate, is coupled to a radioisotope or enzymatic label such that binding of the compound, e.g., the substrate, to the GRIM-19 polypeptide can be determined by detecting the labeled compound, e.g., substrate, in a complex. For example, compounds can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

In yet another embodiment, a cell-free assay is provided in which a GRIM-19 polypeptide or a biologically active portion thereof is contacted with a test compound and the ability of the test compound to bind to the polypeptide thereof is evaluated. Cell-free assays involve preparing a reaction mixture of the target gene protein and the test compound under conditions and for a time sufficient to allow the two components to interact and bind, thus forming a complex that can be removed and/or detected.

The interaction between two molecules can also be detected, e.g., using fluorescence energy transfer (FET) (see, for example, Lakowicz et al., U.S. Pat. No. 5,631,169; Stavrianopoulos et al., U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determining the ability of a test compound to bind to a GRIM-19 polypeptide can be accomplished using real-time Biomolecular Interaction Analysis (BIA) (see, e.g., Sjolander, S, and Urbaniczky, C., Anal. Chem. 63:2338-2345, 1991; and Szabo et al., Curr. Opin. Struct. Biol. 5:699-705, 1995). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal that can be used as an indication of real-time reactions between biological molecules.

In one embodiment, the sample comprising the GRIM-19 polypeptide or the test compound is anchored onto a solid phase. GRIM-19/test compound complexes anchored on the solid phase can be detected at the end of the reaction.

It may be desirable to immobilize either the GRIM-19 polypeptide, an anti-GRIM-19 polypeptide antibody or its target molecule to facilitate separation of complexed from uncomplexed forms of one or both of the proteins, as well as to accommodate automation of the assay. Binding of a test compound to a GRIM-19 polypeptide, or interaction of a GRIM-19 polypeptide with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-5-transferase/GRIM-19 polypeptide fusion proteins can be adsorbed onto glutathione sepharose beads (Sigma Chemical, St. Louis, Mo.) or glutathione derivatized microtiter plates, which are then combined with the test compound or the test compound and a sample comprising the GST-tagged GRIM-19 polypeptide, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above.

Other techniques for immobilizing a complex of GRIM-19 polypeptides on matrices include using conjugation of biotin and streptavidin. For example, biotinylated proteins can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

In one embodiment, this assay is performed utilizing antibodies reactive with an epitope on the GRIM-19 polypeptide, but that do not interfere with binding of the GRIM-19 polypeptide to a test compound. Such antibodies can be derivatized to the wells of the plate, and unbound target or GRIM-19 trapped in the wells by antibody conjugation. Methods for detecting such complexes, in addition to those described above for the GST-immobilized complexes, include immunodetection of complexes using antibodies reactive with a component of the GRIM-19 polypeptide, as well as enzyme-linked assays which rely on detecting an enzymatic activity associated with GRIM-19.

Alternatively, cell free assays can be conducted in a liquid phase. In such an assay, the reaction products are separated from unreacted components, by any of a number of standard techniques, including but not limited to: differential centrifugation (see, for example, Rivas, G., and Minton, A. P., Trends Biochem Sci 18:284-7, 1993); chromatography (gel filtration chromatography, ion-exchange chromatography); electrophoresis and immunoprecipitation (see, for example, Ausubel, F. et al., eds. (1999) Current Protocols in Molecular Biology, J. Wiley: New York). Such resins and chromatographic techniques are known to one skilled in the art (see, e.g., Heegaard, N. H., J Mol Recognit 11:141-8, 1998; Hage, D. S., and Tweed, S. A., J Chromatogr B Biomed Sci Appl. 699:499-525, 1997). Further, fluorescence energy transfer may also be conveniently utilized, as described herein, to detect binding without further purification of the complex from solution. Preferably, cell free assays preserve the structure of the GRIM-19 polypeptide, e.g., by including a membrane component or synthetic membrane components.

In a specific embodiment, the assay includes contacting the GRIM-19 polypeptide or biologically active portion thereof with a known compound which binds the GRIM-19 polypeptide to form an assay mixture, contacting the assay mixture with a test compound, and determining the ability of the test compound to interact with a GRIM-19 polypeptide, wherein determining the ability of the test compound to interact with a GRIM-19 polypeptide includes determining the ability of the test compound to preferentially bind to the GRIM-19 polypeptide, or to modulate the activity of the GRIM-19 polypeptide, as compared to the known compound.

Compounds isolated by this method (or any other appropriate method) may, if desired, be further purified (e.g., by high performance liquid chromatography). In addition, these candidate compounds may be tested for their ability to increase the activity of a GRIM-19 polypeptide (e.g., as described herein). Compounds isolated by this approach may also be used, for example, as therapeutics to treat an intestinal inflammation, inflammatory bowel disease or pathogen infection in a subject. Compounds that are identified as binding to a polypeptide of the invention with an affinity constant less than or equal to 10 mM are considered particularly useful in the invention. Alternatively, any in vivo protein interaction detection system, for example, any two-hybrid assay may be utilized.

In another embodiment, a candidate compound is tested for its ability to enhance the biological activity of a GRIM-19 or NOD2 polypeptide. The biological activity of GRIM-19 or NOD2 polypeptide is assayed using any standard method. For example, GRIM-19 biological activity is assayed using an NFκB activity assay, NOD2 binding assay, or assays for anti-bacterial activity (e.g., bacterial invasion assays and nondestructive bioluminescence cytotoxicity assays). GRIM-19 or NOD2 biological activity in the presence of the compound is compared with a reference value for GRIM-19 or NOD2 biological activity in the absence of the compound.

In another embodiment, a GRIM-19 or NOD2 nucleic acid described herein is expressed as a transcriptional or translational fusion with a detectable reporter, and expressed in an isolated cell (e.g., mammalian or insect cell) under the control of an endogenous or a heterologous promoter. The cell expressing the fusion protein is then contacted with a candidate compound, and the expression of the detectable reporter in that cell is compared to the expression of the detectable reporter in an untreated control cell. A candidate compound that increases the expression of the detectable reporter is a compound that is useful for the treatment of an intestinal inflammation, inflammatory bowel disease or pathogen infection. In preferred embodiments, the candidate compound increases the expression of a reporter gene fused to a GRIM-19 nucleic acid molecule.

One skilled in the art appreciates that the effects of a candidate compound on GRIM-19 expression or biological activity are typically compared to the expression or activity of GRIM-19 in the absence of the candidate compound. Thus, the screening methods include comparing the value of a cell modulated by a candidate compound to a reference value of an untreated control cell.

Expression levels can be compared by procedures well known in the art such as RT-PCR, Northern blotting, Western blotting, flow cytometry, immunocytochemistry, binding to magnetic and/or antibody-coated beads, in situ hybridization, fluorescence in situ hybridization (FISH), flow chamber adhesion assay, and ELISA, microarray analysis, or colorimetric assays, such as the Bradford Assay and Lowry Assay,

Changes in tissue or organ morphology as a result of inflammation further comprise values and/or profiles that can be assayed by methods of the invention by any method known in the art, including x-ray, sonogram and ultrasound.

Molecules that increase GRIM-19 expression or activity include organic molecules, peptides, peptide mimetics, polypeptides, nucleic acids, and antibodies that bind to a GRIM-19 nucleic acid sequence or polypeptide and increase its expression or biological activity are preferred.

A GRIM-19 encoding nucleic acid sequence may also be used in the discovery and development of a therapeutic compound for the treatment of an intestinal inflammation, inflammatory bowel disease or pathogen infection. The encoded protein, upon expression, can be used as a target for the screening of drugs. Additionally, the DNA sequences encoding the amino terminal regions of the encoded protein or Shine-Delgarno or other translation facilitating sequences of the respective mRNA can be used to construct sequences that promote the expression of the coding sequence of interest. Such sequences may be isolated by standard techniques (Ausubel et al., supra).

Small molecules of the invention preferably have a molecular weight below 2,000 daltons, more preferably between 300 and 1,000 daltons, and most preferably between 400 and 700 daltons. It is preferred that these small molecules are organic molecules.

### Animal Models of Inflammatory Bowel Disease

Optionally, compounds identified using screening methods of the invention are characterized for efficacy in animal models of intestinal inflammation. Such animal models are known to the skilled artisan, and include, for example, the severe combined immunodeficient (SCID) mouse model of colitis (Whiting et al., Inflamm Bowel Dis. 4:340-349, 2005), mdr1a−/− mouse model of spontaneous colitis (Wilk et al., Immunol Res. 31:151-160, 2005), the TGF-β1 transfected mice (Valiance et al., Am J Physiol Gastrointest Liver Physiol. Mar. 18, 2005 [Epub ahead of print]), as well as animal models where colitis or intestinal inflammation is induced by treating the animal with a chemical agent, such as trinitrobenzenesulphonic acid, dextran sulphate sodium (DSS), or acetic acid.

### Test Compounds and Extracts

In general, compounds capable of increasing the expression or activity of GRIM-19 polypeptide are identified from large libraries of both natural product or synthetic (or semi-synthetic) extracts or chemical libraries or from polypeptide or nucleic acid libraries, according to methods known in the art. Those skilled in the field of drug discovery and development will understand that the precise source of test extracts or compounds is not critical to the screening procedure(s) of the invention. Compounds used in screens may include known compounds (for example, known therapeutics used for other diseases or disorders). Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds.

Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, chemical compounds to be used as candidate compounds can be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994. Furthermore, if desired, any library or compound is readily modified using standard chemical, physical, or biochemical methods.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:6378-6382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity should be employed whenever possible.

When a crude extract is found to increase the activity of a GRIM-19 polypeptide, or to bind a GRIM-19 polypeptide, further fractionation of the positive lead extract is necessary to isolate chemical constituents responsible for the observed effect. Thus, the goal of the extraction, fractionation, and purification process is the careful characterization and identification of a chemical entity within the crude extract that increases the activity of a GRIM-19 polypeptide. Methods of tractionation and purification of such heterogenous extracts are known in the art. If desired, compounds shown to be useful as therapeutics for the treatment of an intestinal inflammation, inflammatory bowel disease or pathogen infection are chemically modified according to methods known in the art.

### Pharmaceutical Therapeutics

The invention provides a simple means for identifying compositions (including nucleic acids, peptides, small molecule inhibitors, and mimetics) capable of acting as therapeutics for the treatment of an intestinal inflammation, inflammatory bowel disease or pathogen infection. Accordingly, a chemical entity discovered to have medicinal value using the methods described herein is useful as a drug or as information for structural modification of existing compounds, e.g., by rational drug design. Such methods are useful for screening compounds having an effect on a variety of mental conditions characterized by a decrease in the expression of a GRIM-19 gene.

For therapeutic uses, the compositions or agents identified using the methods disclosed herein may be administered systemically, for example, formulated in a pharmaceutically acceptable buffer such as physiological saline. Preferable routes of administration include, for example, oral, topical, enema, subcutaneous, intravenous, interperitoneally, intramuscular, intradermal injections that provide continuous, sustained levels of the drug in the patient. Treatment of human patients or other animals will be carried out using a therapeutically effective amount of an intestinal inflammation, inflammatory bowel disease or pathogen infection therapeutic in a physiologically acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the intestinal inflammation or inflammatory bowel disease. Generally, amounts will be in the range of those used for other agents used in the treatment of other diseases associated with an intestinal inflammation, inflammatory bowel disease or pathogen infection, although in certain instances lower amounts will be needed because of the increased specificity of the compound. A compound is administered at a dosage that controls the clinical or physiological symptoms of an intestinal inflammation, inflammatory bowel disease or pathogen infection as determined by a diagnostic method known to one skilled in the art, or using any that assay that measures the expression or the biological activity of a GRIM-19 polypeptide.

### Formulation of Pharmaceutical Compositions

The administration of a compound for the treatment of an intestinal inflammation, inflammatory bowel disease or pathogen infection may be by any suitable means that results in a concentration of the therapeutic that, combined with other components, is effective in ameliorating, reducing, or stabilizing an intestinal inflammation, inflammatory bowel disease or pathogen infection. The compound may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in the central nervous system or cerebrospinal fluid; (v) formulations that allow for convenient dosing, such that doses are administered, for example, once every one or two weeks; and (vi) formulations that target an intestinal inflammation, inflammatory bowel disease or pathogen infection by using carriers or chemical derivatives to deliver the therapeutic agent to a particular cell type (e.g., intestinal epithelial cell) whose function is perturbed in an intestinal inflammation, inflammatory bowel disease or pathogen infection. For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

### Parenteral Compositions

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active inflammatory bowel disorder therapeutic (s), the composition may include suitable parenterally acceptable carriers and/or excipients. The active inflammatory bowel disorder therapeutic (s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

As indicated above, the pharmaceutical compositions according to the invention may be in the form suitable for sterile injection. To prepare such a composition, the suitable active inflammatory bowel disorder therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

### Controlled Release Parenteral Compositions

Controlled release parenteral compositions may be in form of aqueous suspensions, microspheres, microcapsules, magnetic microspheres, oil solutions, oil suspensions, or emulsions. Alternatively, the active drug may be incorporated in biocompatible carriers, liposomes, nanoparticles, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutam-nine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

### Solid Dosage Forms for Oral Use

Formulations for oral use include tablets containing the active ingredient(s) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug in a predetermined pattern (e.g., in order to achieve a controlled release formulation) or it may be adapted not to release the active drug until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material such as, e.g., glyceryl monostearate or glyceryl distearate may be employed.

The solid tablet compositions may include a coating adapted to protect the composition from unwanted chemical changes, (e.g., chemical degradation prior to the release of the active inflammatory bowel disease therapeutic substance). The coating may be applied on the solid dosage form in a similar manner as that described in Encyclopedia of Pharmaceutical Technology, supra.

At least two active inflammatory bowel disorder therapeutics may be mixed together in the tablet, or may be partitioned. In one example, the first active inflammatory bowel disorder therapeutic is contained on the inside of the tablet, and the second active inflammatory bowel disorder therapeutic is on the outside, such that a substantial portion of the second active inflammatory bowel disorder therapeutic is released prior to the release of the first active inflammatory bowel disorder therapeutic.

Formulations for oral use may also be presented as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment.

### Controlled Release Oral Dosage Forms

Controlled release compositions for oral use may, e.g., be constructed to release the active inflammatory bowel disorder therapeutic by controlling the dissolution and/or the diffusion of the active substance. Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

A controlled release composition containing one or more therapeutic compounds may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the compound(s) with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

### Treatment of an Inflammatory Bowel Disease

Given that decreased GRIM-19 expression is observed in tissues affected by an inflammatory bowel disease, compositions and methods that increase the expression, activity, or local concentration of a GRIM-19 polypeptide can prevent or ameliorate an intestinal inflammation, inflammatory bowel disease or pathogen infection characterized by inadequate GRIM-19 expression or activity.

In one example, a GRIM-19 polypeptide is provided in a pharmaceutical composition such that it is effective for the treatment of an intestinal inflammation, inflammatory bowel disease or pathogen infection. Methods for providing protein therapeutics to an intestinal epithelial cell are described, for example, in U.S. Pat. No. 6,455,042. A GRIM-19 polypeptide can be provided either directly (e.g., by administration to the intestine) or systemically (for example, by any conventional recombinant protein administration technique). The dosage of the administered protein depends on a number of factors, including the size and health of the individual patient. For any particular subject, the specific dosage regimes should be adjusted over time according to the individual need and the professional judgment of the person administering or supervising the administration of the compositions. Generally, between 0.1 mg and 100 mg, is administered per day to an adult in any pharmaceutically acceptable formulation.

In another embodiment, a therapeutic gene product is delivered to cells that line the lumen of the gastrointestinal tract. A transforming formulation comprising a GRIM-19 encoding nucleic acid molecule is introduced into the gastrointestinal tract (e.g., via the mouth) where it is absorbed into cells lining the lumen of the gastrointestinal tract. The DNA is then expressed within these cells. The transformed intestinal cells then express a protein encoded by GRIM-19 nucleic acid molecule and secrete a therapeutically effective amount of the protein into the bloodstream or into the gastrointestinal tract via natural secretory pathways. Preferably, the intestinal cell into which the DNA of interest is introduced and expressed is an epithelial cell of the intestine, and may be an intestinal cell of either the small or large intestine. The nucleic acid molecule is delivered to those cells in a form in which it can be taken up by the cells such that sufficient levels of protein can be produced to increase, for example, an innate mucosal response to a pathogen or to decrease intestinal inflammation. Methods of transforming an intestinal epithelial cell with a nucleic acid molecule are known in the art, and are described, for example, in U.S. Pat. Nos. 6,831,070 and 6,455,042 and in U.S. Published Patent Application Nos. 20040115254 and 20050026863.

### Formulations

A GRIM-19-encoding nucleic acid molecule can be formulated as a DNA- or RNA-liposome complex formulation. Such complexes comprise a mixture of lipids that bind to genetic material (DNA or RNA), providing a hydrophobic core and hydrophilic coat that allows the genetic material to be delivered into cells. Liposomes that can be used in accordance with the invention include DOPE (dioleyl phosphatidyl ethanol amine), CUDMEDA (N-(5-cholestrum-3-.beta.-ol 3-urethanyl)-N′,N′-dimethylethylene diamine). Of particular interest is the use of the cationic transport reagents and polyfunctional cationic cytofectins described in U.S. Pat. No. 5,527,928 and PCT Published Application Nos. WO 96/10555 and WO 97/11935.

Other formulations can also be used in accordance with the present invention. Such formulations include DNA or RNA coupled to a carrier molecule (e.g., an antibody or a, receptor ligand) that facilitates delivery to intestinal epithelial cells for the purpose of altering the biological properties of the cells. Exemplary protein carrier molecules include antibodies specific to the cells of a targeted intestinal cell or receptor ligands, i.e., molecules capable of interacting with receptors associated with a cell of a targeted intestinal cell.

In one embodiment, the formulation is primarily composed of naked DNA (e.g., DNA that is not contained within a viral vector) and/or is substantially free of detergent (e.g., ionic and nonionic detergents, e.g., polybrene, etc.) or mucolytic agents (e.g., N-acetylcysteine, dithiothreitol, and pepsin). Non-viral approaches can also be employed for the introduction of a therapeutic to a cell of a patient having an intestinal inflammation, inflammatory bowel disease or pathogen infection. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Preferably the nucleic acids are administered in combination with a liposome and protamine.

cDNA expression for use in such methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types, such as an intestinal epithelial cell, can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

For example, where the targeted intestinal cell is a small intestine epithelial cell and the nucleic acid is administered orally as naked DNA, the naked DNA is administered at a concentration sufficient to reach the small intestine to provide a DNA concentration effective to transform the targeted small intestine epithelial cells and provide for therapeutic levels of the protein in either the blood or the gastrointestinal tract. In general, the nucleic acid is administered ranging from about 1 mg to 1 gram, generally about 100 mg to about 1 gram, depending on the formulation used. In general, dosages for humans are approximately 200 times dosages effective in a rat or mouse model.

### Combination Therapies

Optionally, an intestinal inflammation or inflammatory bowel disease therapeutic may be administered in combination with any other standard active inflammatory bowel disorder therapy; such methods are known to the skilled artisan and described in Remington's Pharmaceutical Sciences by E. W. Martin. An intestinal inflammation or inflammatory bowel disease therapeutic of the invention may be administered in combination with any standard or experimental therapy useful for treating an intestinal inflammation or inflammatory bowel disorder. Such therapies include, but are not limited to, methods for controlling inflammation containing mesalamine, (e.g., Sulfasalazine, Asacol, Dipentum, or Pentasa) or Natalizumab, corticosteroids (e.g., budesonide), immunosuppressive agents (e.g., methotrexate, cyclosporine 6-mercaptopurine and azathioprine), anti-tumor necrosis factor agents (e.g., infliximab), antibiotics (e.g., ampicillin, sulfonamide, cephalosporin, tetracycline, or metronidazole), antidiarrheal agents (e.g., diphenoxylate, loperamide, and codeine), and nutritional supplements.

### Patient Monitoring

The disease state or treatment of a patient having an intestinal inflammation or inflammatory bowel disease can be monitored using the methods and compositions of the invention. In some embodiments, quantitative real-time PCR is used to assay the expression profile of a GRIM-19 nucleic acid molecule. Such monitoring may be useful, for example, in assessing the efficacy of a particular drug or treatment regimen. Therapeutics that increase the expression of a GRIM-19 nucleic acid molecule or polypeptide are taken as particularly useful in the invention.

### Anti-GRIM-19 and NOD2 Antibodies

Antibodies that specifically bind to a GRIM-19 or NOD2 polypeptide are also useful in the methods of the invention. As used herein, the term “antibody” means not only intact antibody molecules but also fragments of antibody molecules retaining immunogen-binding ability. Such fragments are also well known in the art and are regularly employed both in vitro and in vivo. Accordingly, the term “antibody” means not only intact immunoglobulin molecules but also the well-known active fragments F(ab′)₂, and Fab. F(ab′)₂, and Fab fragments which lack the Fc fragment of intact antibody, clear more rapidly from the circulation, and may have less non-specific tissue binding of an intact antibody (Wahl et al., J. Nucl. Med. 24:316-325 (1983). The antibodies of the invention comprise whole native antibodies, bispecific antibodies; chimeric antibodies; Fab, Fab′, single chain V region fragments (scFv) and fusion polypeptides. Preferably, the antibodies of the invention are monoclonal. Alternatively the antibody may be a polyclonal antibody. The preparation and use of polyclonal antibodies is also known to one of ordinary skill in the art. The invention also encompasses hybrid antibodies, in which one pair of heavy and light chains is obtained from a first antibody, while the other pair of heavy and light chains is obtained from a different second antibody. Such hybrids may also be formed using humanized heavy and light chains. Such antibodies are often referred to as “chimeric” antibodies.

In general, intact antibodies are said to contain “Fc” and “Fab” regions. The Fc regions are involved in complement activation and are not involved in antigen binding. An antibody from which the Fc′ region has been enzymatically cleaved, or which has been produced without the Fc′ region, designated an “F(ab′)₂” fragment, retains both of the antigen binding sites of the intact antibody. Similarly, an antibody from which the Fc region has been enzymatically cleaved, or which has been produced without the Fc region, designated an “Fab′” fragment, retains one of the antigen binding sites of the intact antibody. Fab′ fragments consist of a covalently bound antibody light chain and a portion of the antibody heavy chain, denoted “Fd.” The Fd fragments are the major determinants of antibody specificity (a single Fd fragment may be associated with up to ten different light chains without altering antibody specificity). Isolated Fd fragments retain the ability to specifically bind to immunogenic epitopes.

Antibodies can be made by any of the methods known in the art utilizing GRIM-19 or NOD2, or immunogenic fragments thereof, as an immunogen. One method of obtaining antibodies is to immunize suitable host animals with an immunogen and to follow standard procedures for polyclonal or monoclonal production. The immunogen will facilitate presentation of the immunogen on the cell surface. Immunization of a suitable host can be carried out in a number of ways. Nucleic acid sequences encoding GRIM-19 or NOD2, or immunogenic fragments thereof, can be provided to the host in a delivery vehicle that is taken up by immune cells of the host. The cells will in turn express the receptor on the cell surface generating an immunogenic response in the host. Alternatively, nucleic acid sequences encoding GRIM-19 or NOD2, or immunogenic fragments thereof, can be expressed in cells in vitro, followed by isolation of the receptor and administration of the receptor to a suitable host in which antibodies are raised.

Using either approach, antibodies can then be purified from the host. Antibody purification methods may include salt precipitation (for example, with ammonium sulfate), ion exchange chromatography (for example, on a cationic or anionic exchange column preferably run at neutral pH and eluted with step gradients of increasing ionic strength), gel filtration chromatography (including gel filtration HPLC), and chromatography on affinity resins such as protein A, protein G, hydroxyapatite, and anti-immunoglobulin.

Antibodies can be conveniently produced from hybridoma cells engineered to express the antibody. Methods of making hybridomas are well known in the art. The hybridoma cells can be cultured in a suitable medium, and spent medium can be used as an antibody source. Polynucleotides encoding the antibody of interest can in turn be obtained from the hybridoma that produces the antibody, and then the antibody may be produced synthetically or recombinantly from these DNA sequences. For the production of large amounts of antibody, it is generally more convenient to obtain an ascites fluid. The method of raising ascites generally comprises injecting hybridoma cells into an immunologically naive histocompatible or immunotolerant mammal, especially a mouse. The mammal may be primed for ascites production by prior administration of a suitable composition; e.g., Pristane.

Monoclonal antibodies (Mabs) produced by methods of the invention can be “humanized” by methods known in the art. “Humanized” antibodies are antibodies in which at least part of the sequence has been altered from its initial form to render it more like human immunoglobulins. Techniques to humanize antibodies are particularly useful when non-human animal (e.g., murine) antibodies are generated. Examples of methods for humanizing a murine antibody are provided in U.S. Pat. Nos. 4,816,567, 5,530,101, 5,225,539, 5,585,089, 5,693,762 and 5,859,205. In one another version, the heavy chain and light chain C regions are replaced with human sequence. In another version, the CDR regions comprise amino acid sequences for recognition of antigen of interest, while the variable framework regions have also been converted to human sequences. See, for example, EP 0329400. It is well established that non-CDR regions of a mammalian antibody may be replaced with corresponding regions of non-specific or hetero-specific antibodies while retaining the epitope specificity of the original antibody. This technique is useful for the development and use of humanized antibodies in which non-human CDRs are covalently joined to human FR and/or Fc/pFc′ regions to produce a functional antibody. In a third version, variable regions are humanized by designing consensus sequences of human and mouse variable regions, and converting residues outside the CDRs that are different between the consensus sequences.

Construction of phage display libraries for expression of antibodies, particularly the Fab or scFv portion of antibodies, is well known in the art (Heitner, 2001). The phage display antibody libraries that express antibodies can be prepared according to the methods described in U.S. Pat. No. 5,223,409 incorporated herein by reference. Procedures of the general methodology can be adapted using the present disclosure to produce antibodies of the present invention. The method for producing a human monoclonal antibody generally involves (1) preparing separate heavy and light chain-encoding gene libraries in cloning vectors using human immunoglobulin genes as a source for the libraries, (2) combining the heavy and light chain encoding gene libraries into a single dicistronic expression vector capable of expressing and assembling a heterodimeric antibody molecule, (3) expressing the assembled heterodimeric antibody molecule on the surface of a filamentous phage particle, (4) isolating the surface-expressed phage particle using immunoaffinity techniques such as panning of phage particles against a preselected immunogen, thereby isolating one or more species of phagemid containing particular heavy and light chain-encoding genes and antibody molecules that immunoreact with the preselected immunogen. The preselected immunogen can be provided by or obtained from cells of the invention that express GRIM-19 or NOD2, or immunogenic fragments thereof, on the cell surface.

Single chain variable region fragments are made by linking light and heavy chain variable regions by using a short linking peptide. Any peptide having sufficient flexibility and length can be used as a linker in a scFv. Usually the linker is selected to have little to no immunogenicity. An example of a linking peptide is (GGGGS)₃, which bridges approximately 3.5 nm between the carboxy terminus of one variable region and the amino terminus of another variable region. Other linker sequences can also be used. All or any portion of the heavy or light chain can be used in any combination. Typically, the entire variable regions are included in the scFv. For instance, the light chain variable region can be linked to the heavy chain variable region. Alternatively, a portion of the light chain variable region can be linked to the heavy chain variable region, or a portion thereof. Compositions comprising a biphasic scFv could be constructed in which one component is a polypeptide that recognizes an immunogen and another component is a different polypeptide that recognizes a different antigen, such as a T cell epitope.

ScFvs can be produced either recombinantly or synthetically. For synthetic production of scFv, an automated synthesizer can be used. For recombinant production of scFv, a suitable plasmid containing a polynucleotide that encodes the scFv can be introduced into a suitable host cell, either eukaryotic, such as yeast, plant, insect or mammalian cells, or prokaryotic, such as Escherichia coli, and the protein expressed by the polynucleotide can be isolated using standard protein purification techniques.

A particularly useful system for the production of scFvs is plasmid pET-22b(+) (Novagen, Madison, Wis.) in E. coli. pET-22b(+) contains a nickel ion binding domain consisting of 6 sequential histidine residues, which allows the expressed protein to be purified on a suitable affinity resin. Another example of a suitable vector for the production of scFvs is pcDNA3 (Invitrogen, San Diego, Calif.) in mammalian cells, described above.

Expression conditions should ensure that the scFv assumes functional and, preferably, optimal tertiary structure. Depending on the plasmid used (especially the activity of the promoter) and the host cell, it may be necessary or useful to modulate the rate of production. For instance, use of a weaker promoter, or expression at lower temperatures, may be necessary or useful to optimize production of properly folded scFv in prokaryotic systems; or, it may be preferable to express scFv in eukaryotic cells.

### Diagnostics

Expression levels of a GRIM-19 nucleic acid molecule or polypeptide may be correlated with a particular disease state, and thus are useful in diagnosis. In one embodiment, a patient having an intestinal inflammation or inflammatory bowel disease will show a decrease in the expression of a GRIM-19 nucleic acid molecule. Alterations in gene expression are detected using methods known to the skilled artisan and described herein. In another embodiment, oligonucleotides or longer fragments derived from a GRIM-19 nucleic acid may be used as a targets to identify genetic variants, mutations, and polymorphisms. Such information can be used to diagnose an intestinal inflammation or inflammatory bowel disease. In another embodiment, an alteration in the expression of a GRIM-19 nucleic acid molecule is detected using real-time quantitative PCR (Q-rt-PCR) to detect changes in gene expression.

In another embodiment, an antibody that specifically binds a GRIM-19 polypeptide may be used for the diagnosis of an intestinal inflammation or inflammatory bowel disease. A variety of protocols for measuring an alteration in the expression of such polypeptides are known, including immunological methods (such as ELISAs and RIAs), and provide a basis for diagnosing an intestinal inflammation or inflammatory bowel disease. Again, a decrease in the level of the polypeptide is diagnostic of a patient having an intestinal inflammation or inflammatory bowel disease.

In yet another embodiment, hybridization with PCR probes that are capable of detecting a GRIM-19 nucleic acid molecule, including genomic sequences, or closely related molecules, may be used to hybridize to a nucleic acid sequence derived from a patient having an intestinal inflammation or inflammatory bowel disease. The specificity of the probe determines whether the probe hybridizes to a naturally occurring sequence, allelic variants, or other related sequences. Hybridization techniques may be used to identify mutations indicative of an intestinal inflammation or inflammatory bowel disease, or may be used to monitor expression levels of these genes (for example, by Northern analysis (Ausubel et al., supra).

In yet another approach, humans may be diagnosed for a propensity to develop an intestinal inflammation or inflammatory bowel disease by direct analysis of the sequence of a GRIM-19 nucleic acid molecule. The sequence of a GRIM-19 nucleic acid molecule derived from a subject is compared to a reference sequence. An alteration in the sequence of the GRIM-19 nucleic acid molecule indicates that the patient has or has a propensity to develop an intestinal inflammation or inflammatory bowel disease.

### Kits

The invention also provides kits for the treatment or prevention of an intestinal inflammation, inflammatory bowel disease or pathogen infection. In one embodiment, the kit includes an effective amount of a compound herein in unit dosage form, together with instructions for administering the compound to a subject suffering from or susceptible to an intestinal inflammation or inflammatory bowel disorder. In other embodiments, the kit comprises a sterile container which contains the compound; such containers can be boxes, ampules, bottles, vials, tubes, bags, pouches, blister-packs, or other suitable container form known in the art. Such containers can be made of plastic, glass, laminated paper, metal foil, or other materials suitable for holding medicaments. The instructions will generally include information about the use of the compound of the formulae herein for treatment of an intestinal inflammation or inflammatory bowel disorder thereof. In other embodiments, the instructions include at least one of the following: description of the compound; dosage schedule and administration for treatment an intestinal inflammation or inflammatory bowel disorder; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The invention also provides kits for the diagnosis of an intestinal inflammation or inflammatory bowel disease. In one embodiment, the kit detects a decrease in the expression of a GRIM-19 nucleic acid molecule or polypeptide relative to a reference level of expression. In another embodiment, the kit detects an alteration in the sequence of a GRIM-19 nucleic acid molecule derived from a subject relative to a reference sequence. In related embodiments, the kit includes agents for monitoring the expression of a GRIM-19 nucleic acid molecule, such as primers or probes that hybridize to a GRIM-19 nucleic acid molecule. In other embodiments, the kit includes an antibody that binds to a GRIM-19 polypeptide. Optionally, the kit includes directions for monitoring GRIM-19 expression or polypeptide levels.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

Innate immunity relies on the expression of Toll-like receptors, which are a family of trans-membrane proteins, and Nod proteins, which are a family of intracellular bacterial sensor proteins that are able to recognize highly conserved microbial motifs (1), that are key components of innate immunity. The NOD2 gene is the first susceptibility gene associated with Crohn's disease (2, 3). NOD2, which is also known as CARD15/NOD2, is located on chromosome 16q12. The NOD2 protein includes N-terminal CARD domains, a nucleotide-binding domain (NBD), and multiple C-terminal leucine-rich repeat (LRR) regions (4).

NOD2 recognizes muramyl dipeptide (MDP-LD) and subsequently activates NF-κB through a pathway that involves RIP2/RICK and members of the Toll-like receptor-sensing cascade (8-13). The NF-κB pathway is a proinflammatory signaling pathway. A direct interaction between NOD2 and TGF-β-activated kinase 1 (TAK1) was recently shown and TAK1 regulates NOD2-mediated NF-κB activation (5). In addition, NF-κB activation induced by Streptococcus ppneumoniae depends on NOD2 (6). A recent study of NOD2-deficient mice revealed that they lacked protective immunity in response to bacterial muramyl dipeptide (Science 307:731-734, 2005). In addition, the mice are susceptible to bacterial infection via an oral route. It was previously shown that a mutant CARD15/NOD2 protein, 3020insC, exhibits impaired function as a defensive factor against intracellular bacteria in intestinal epithelial cells (IEC) (19). The studies described in more detail below demonstrated that GRIM-19 is required for NOD2-mediated NF-κB activation and for the anti-bacterial effects of NOD2.

### Example 1

### Human Intestinal Epithelial Cell Lines and Primary Cells Express NOD1/CARD4 and NOD2/CARD15

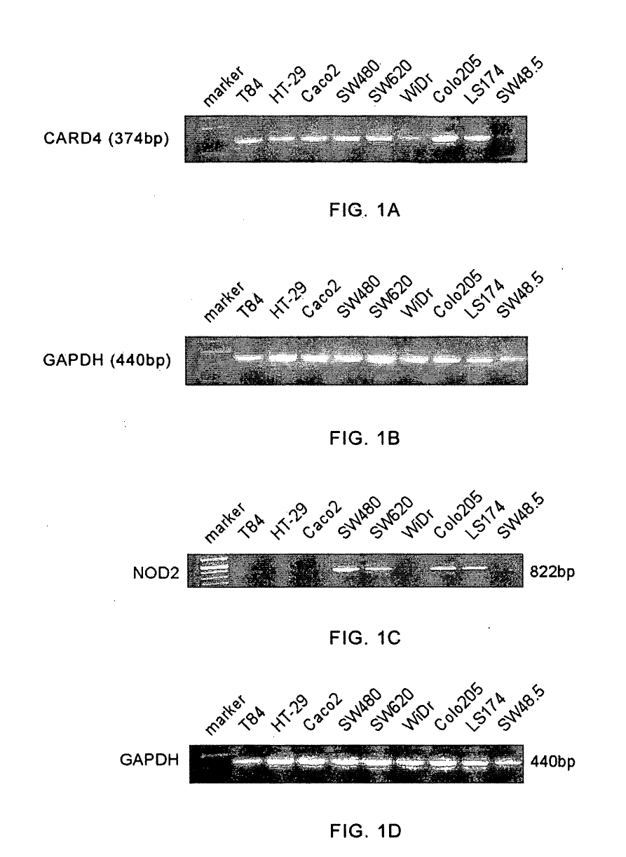

NOD1 and NOD2 expression and their regulation have not been previously demonstrated in IEC lines. Accordingly, expression of NOD1 and NOD2 was assessed by RT-PCR in several independent derived IEC lines: HT-29, T84, Caco2, SW480, SW620, Colo205, WiDr, SW48.5, and LS174. GAPDH (440 bp) was used as internal control. The identity of all fragments was confirmed by sequencing. As shown in FIGS. 1A-1D, NOD1 was constitutively expressed in all IEC lines examined. Although initial reports had concluded on the basis of total tissue Northern blot analysis that NOD2 expression was confined to monocytes in peripheral blood, NOD2 (product size: 822 bp) mRNA was present in several independently derived colonic epithelial lines, including SW480, SW620, T84, colo205, and LS174 cells (FIGS. 1A-1D).

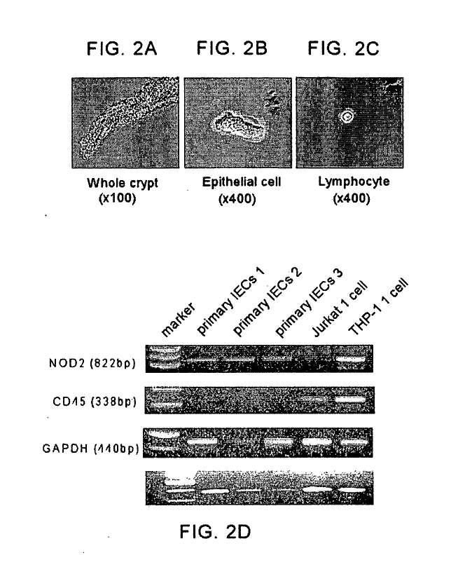

Following demonstration of mRNA expression of NOD1 and NOD2 by IEC lines, expression in primary isolated intestinal epithelial cells was evaluated to assess the relevance of the observations using the in vitro models. Human colonic epithelial cells were isolated from whole crypts obtained from colonic biopsies (FIG. 2A-C) by treatment with 0.3 mM sodium tetraphenylborate in PBS. To avoid contamination by non-epithelial origin cells, isolated epithelial cells were individually picked by micropipetting. Markers for lymphocytes and monocytic cells (CD45 and CD68) were not detected in these isolated intestinal epithelial cells by highly sensitive RT-PCR (data not shown). Total RNA from a single Jurkat cell and THP-1 cell served as positive controls for CD45 and CD68, respectively. As shown in FIG. 2D, NOD1 (374 bp) was expressed in all isolated primary intestinal epithelial cells examined. NOD1 (374 bp) and CARD15/NOD2 (822 bp) mRNA was assayed by RT-PCR in isolated primary intestinal epithelial cells prepared from normal colonic mucosa obtained from five individual patients (primary IECs 1, 2, 3, 4, and 5). Total RNA for each sample was obtained from 10 to 20 isolated primary intestinal epithelial cells. RT-PCR for CD45 and CD68 (data not shown) was performed to confirm the lack of contamination by non-epithelial cells. The sensitivity of RT-PCR of CD45 and CD68 was validated by detection of an appropriate product using total RNA from a single Jurkat cell and THP-1 cell.

### Example 2

### Expression of NODs in IEC is Differentially Regulated by Cytokines

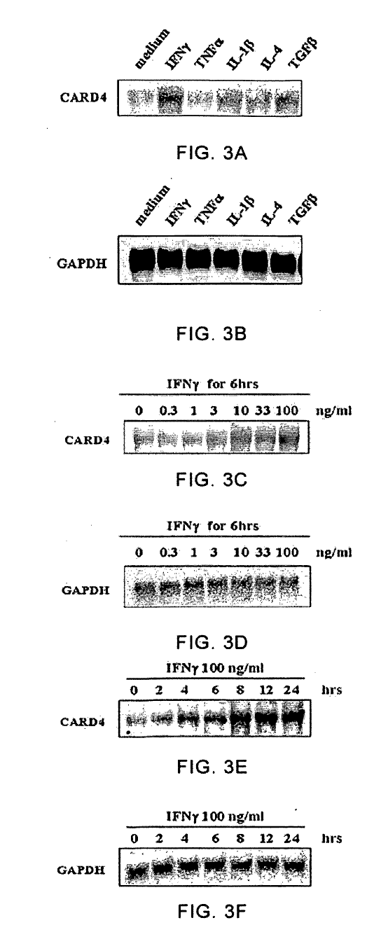

Regulation of NOD1 expression had not been described previously. As shown in FIGS. 3A, 3B, 3C and 3D, IFNγ augmented NOD1 mRNA expression in SW480 cells. Other cytokines examined (TNFβ, IL-β, IL-4, and TGFβ) did not affect NOD1 mRNA expression. The effect of IFNγ on NOD1 mRNA expression is time and concentration dependent manner as assessed by Northern blot analysis (FIG. 3C-3E).

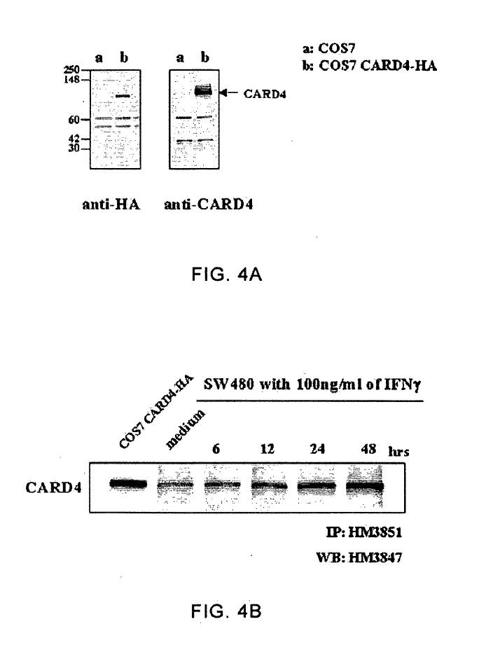

To investigate the expression of NOD1 protein, anti-NOD1 sera were generated. The specificity and the sensitivity were confirmed using lysates from COS7 cells transiently transfected with the HA tagged CARD4 expression plasmid, pCl CARD4-HA (FIG. 4A). Consistent with the regulation of mRNA expression, NOD1 protein was also augmented in SW480 cells by IFNγ stimulation (FIG. 4B).

### Example 3

### IRF-1 is Essential for Up-Regulation of CARD4/NOD1 Transcription by IFNγ

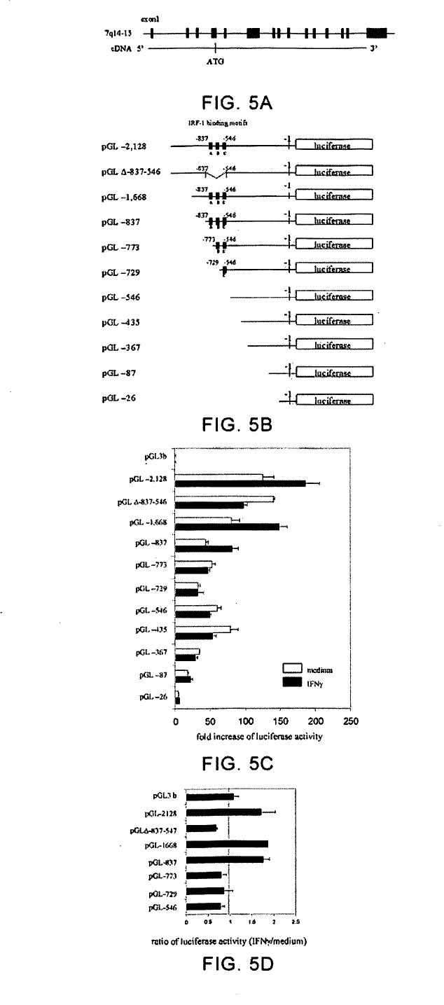

To identify the transcriptional regulation of NOD1, a series of luciferase reporter vectors was constructed, containing up to 2,128 base pairs corresponding to the DNA sequence upstream of base 1 and extending 21 base pairs into the first exon of NOD1 (FIGS. 5A and 5B). Luciferase activity in SW480 cells transfected with a vector containing the entire 2,128 base pairs upstream DNA (pGL-2128) was 125±16 fold higher than that obtained with the empty pGL3 basic vector. Promoter activity was significantly decreased in pGL-26 (4.2±1.4) compared with in pGL-367 (34.4±1.1), indicating that -26 to -367 upstream of exon1 is essential for basal NOD1 expression.

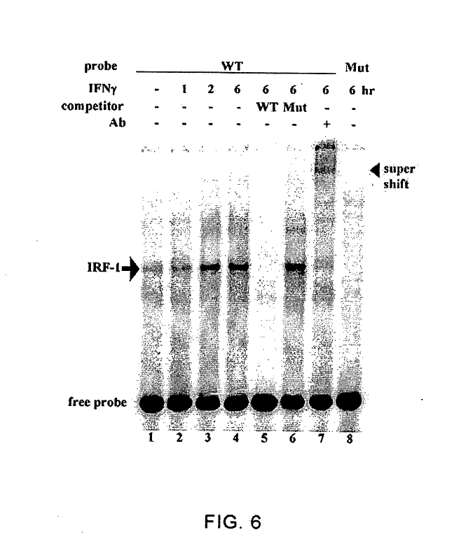

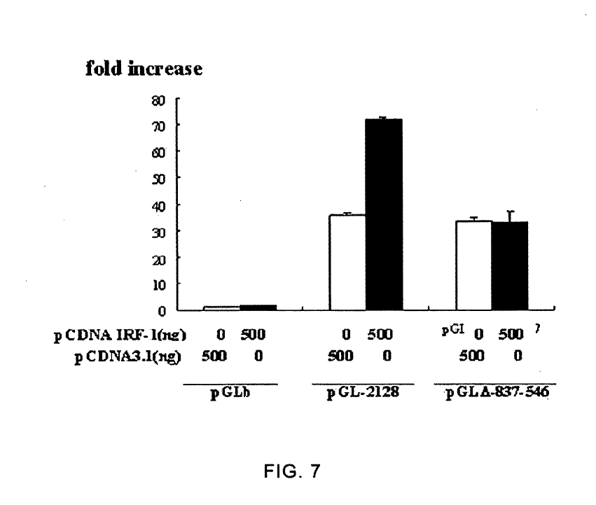

As shown in FIGS. 5C and 5D, IFNγ increased 80% luciferase activity in SW480 cells transfected with pGL-2128. Luciferase activity of cells transfected with deletion constructs, pGLΔ-837-546, pGL-837, and pGL-546, demonstrated that sequences within -837 to -546 of the promoter are essential for activation by IFNγ. Three interferon regulatory factor-1 (IRF-1) binding motifs (IRF-1A; -791 to -782, IRF-1B; -787 to -778, IRF-1B; -694 to -689) (FIGS. 5B and 6A) are clustered in this region. Luciferase activity of cells transfected with pGL-837, pGL-773 and pGL-729 suggested that the most distal IRF-1 binding motif (IRF-1A; -791 to -782) is essential for the IFNγ effect (FIGS. 5C and 5D). Consistent with the promoter analysis, oligonucleotides corresponding to IRF-1 binding sequences (IRF-1 A) in NOD1 promoter specifically bound nuclear IRF-1 protein in IFNγ treated SW480 cells in electrophoretic mobility shift assays (FIG. 6, lanes 1-4). The band reflecting the complex with IRF-1 oligonucleotides was super shifted by anti-IRF-1 antibody (lane 4 and 7). This confirmed that IRF-1 mRNA and nuclear protein expression were rapidly upregulated by IFNγ treatment in SW480 cells. To assess whether over-expressed IRF-1 can activate NOD1 promoter, promoter analysis was performed using SW480 cells co-transfected with IRF-1 expression plasmid. Promoter activity of pGL-2128 was activated 2.0 fold (71.8±0.6vs 35.9±0.9) in SW480 cells co-transfected with pcDNA IRF-1 expression vector but not with pGLΔ-837-546, which lacks the IRF-1 cluster region (FIG. 7). These results suggested that rapid augmentation of nuclear IRF-1 protein by IFNγ treatment results in activation of NOD1 transcription.

### Example 4

### Cytokine Regulation of NOD2 Expression in IEC

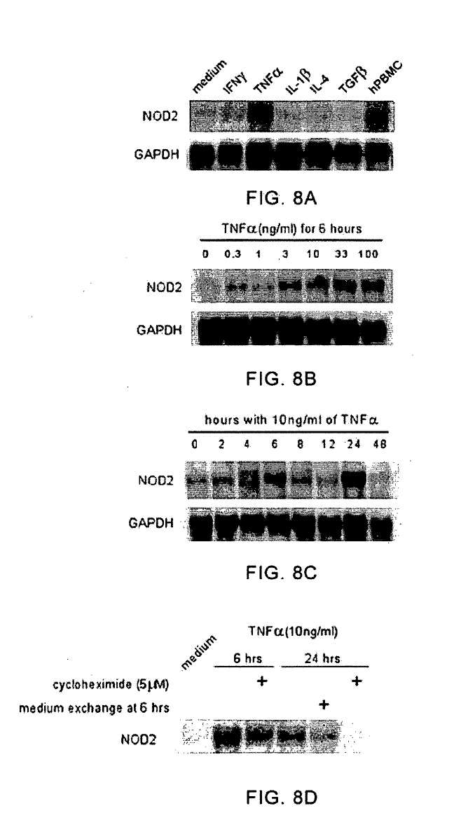

Regulation of NOD2 expression had not been previously characterized. In order to better understand how NOD2 expression might vary in the context of mucosal inflammation, the effects of cytokines on epithelial NOD2 expression were studied using the SW480 cell line. TNFα up-regulated NOD2 mRNA expression in SW480 cells (FIG. 8A), an effect that was concentration and time-dependent (FIGS. 8B and 8C). Interestingly, the kinetics of NOD2 mRNA expression by 10 ng/ml TNFα revealed two peaks (at six hours and twenty-four hours). Cycloheximide, a protein synthesis inhibitor, inhibited the second peak of mRNA expression (at twenty-four hours), but not the first peak (at six hours) suggesting that the second peak of mRNA expression depends upon protein synthesis (FIG. 8D). Therefore, the data suggests that NOD2 in IECs may be modulated by proinflammatory cytokines in intestinal mucosal inflammation and by an innate immune response to microorganisms.

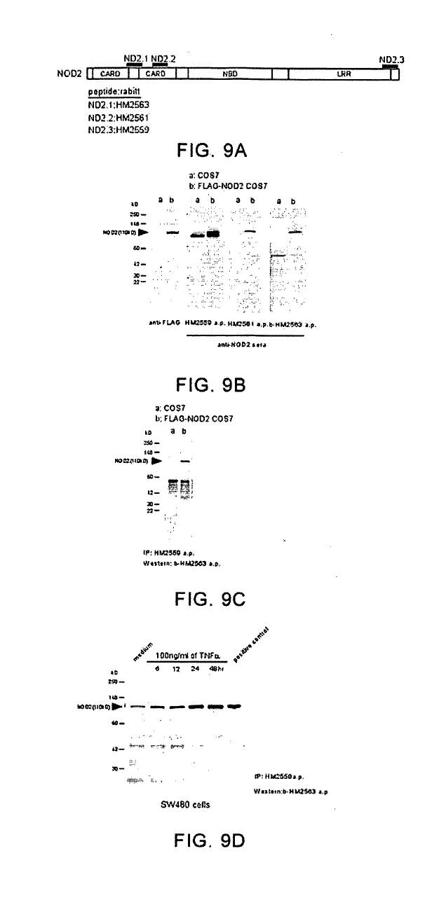

Previous reported efforts had not been successful in detecting active NOD2 protein in vitro or in vivo. To detect the expression of active CARD15/NOD2 protein, anti-NOD2 sera (FIG. 9A) were generated. Using the anti-NOD2 serum, it was possible to demonstrate the presence of NOD2 protein in COS7 cells transiently transfected with pCMV FLAG-NOD2 plasmid by Western blot analysis (FIGS. 9B and 9C). Consistent with mRNA expression, NOD2 protein was upregulated by TNFα stimulation in a time-dependent manner (FIG. 9D). Increased production of TNFα has been noted in the mucosa of patients with Crohn's disease.

### Example 5

### Mutant NOD2 Fails to Restrict Survival of Intracellular Bacteria

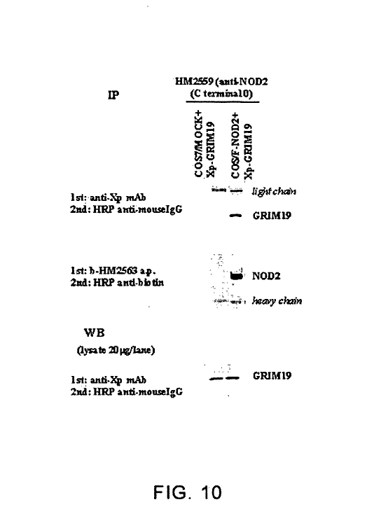

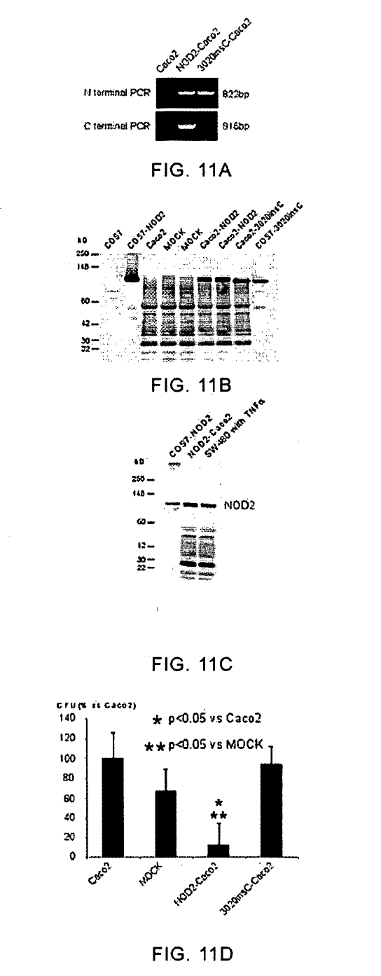

As shown in FIG. 10, the Caco2 cell line does not express endogenous NOD2. To investigate the function of NOD2 in IECs, plasmid constructs of wild type NOD2 and the Crohn's disease associated mutant 3020insC-NOD2 were stably tiansfected into Caco2 (designated NOD2-Caco2, 3020insC-Caco2, respectively). NOD2 serves as a cytosolic LPS receptor, but effects on actual bacterial invasion had not been examined. Expression of protein at levels comparable to that produced by SW480 cells in “physiologic” response to TNF (100 ng/ml) underscoring the relevance of the observed functional effects in the transfectants (FIG. 11C) was confirmed by immunoprecipitation and immunoblotting (FIG. 11B).

To determine whether NOD2/CARD15 protein results in alteration in the functional outcome of bacterial survival, untransfected and transfected IECs (Caco2, MOCK, NOD2-Caco2 and 3020insC-Caco2 cells) were infected with S. typhimurium. Results are shown in FIGS. 11A-11D. Viable intracellular bacteria in cell extracts were then measured using a gentamicin protection assay after removing all residual extracellular bacteria. NOD2-Caco2 cell lines compared to untransfected Caco2 cells and MOCK cell lines (FIG. 11D). The efficacy of the treatment condition in eliminating non-invasive bacteria from the medium was confirmed, using non-pathogenic E. coli F18. The percentage of colony forming unit (CFU) of the latter was less than 1% in both Caco2 and NOD2-Caco2 cells (data not shown). Thus, the bacteria recovered reflected intracellular organisms and diminished CFU in cells with activation of NOD2 reflected effects on intracellular bacteria. In contrast, CFU in cells stably transfected with the 3020insC mutant NOD2 were indistinguishable from untransfected Caco2 and MOCK cells (FIG. 11D). NOD2 not only protects host cells by preventing the survival of S. typhimurium, but also that this host defensive effect of NOD2 is functionally defective in 3020insC-Caco2. Thus, dysfunction of NOD2 can enable bacteria to survive following host-bacteria interaction.

Since the isolation of AIEC from individuals with Crohn's Disease delineation of the IEC response to new classes of enteric bacteria associated with Inflammatory Bowel Disease has been of great interest (1-3). While these bacteria have many properties that resemble “garden variety” commensals, they effect low-level invasion of intestinal epithelial cells. Following studies of the effect of NOD2 on conventional invasive enteric pathogen, such as AIEC and adherent non invasive E. coli as well as other non-pathogenic E. coli, comparable studies were carried out on E. coli associated with Crohn's Disease. The clones exhibit an invasive phenotype, though the overall efficiency of invasion was substantially less than that observed for Salmonella by more than two logs. Further, the expression of NOD2 in IEC by percentage, is even more marked than observed for Salmonella, perhaps reflective of the lesser virulence of the CD associated AIEC. These findings provide further evidence of the relevance of proposed studies to understanding of mucosal homeostasis and its disruption in the pathophysiology of IBD.

### Example 6

### NOD2 is Recruited to the Membrane in IEC and Redistributed after Bacterial Invasion

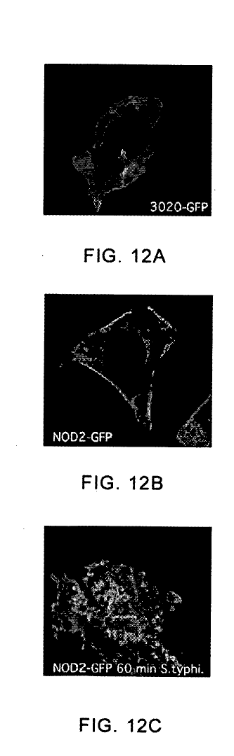

Further characterization of these mechanisms was undertaken to define the subcellular localization of NOD2. Model cells were transfected with FLAG, GFP or myc tagged NOD2 and the protein was then localized by light and confocal microscopy. As shown in FIGS. 12A-12C, GFP tagged NOD2 was initially focally present in the cytoplasm, but associated with the cell membrane. However, following invasion by Salmonella tagged red NOD2 was redistributed away from the membrane appear to coalesce around the pathogen. In contrast the CD mutant NOD2 remained diffuse in the cytoplasm and did not appear to either localize to cell membrane or invading bacteria. These observations suggest specific intracellular structural responses to bacterial infection that yield close physical apposition of NOD2 with the intracellular bacteria.

### Example 7

### NODs Induce a Distinctive Transcriptional Response Following Bacterial Invasion

In preliminary studies, transcriptional responses have been assessed before and following (30 minutes and 120 minutes) invasion of Caco2 cells lacking NOD2 or stably expressing either wild type or mutant (C3020Ins) NOD2 with Salmonella. Expression of RNA was compared in Caco2 with Caco2-NOD2, and Caco2 with Caco2-3020insC, respectively using a microarray format fabricated at the MGH by spotting a set of 70-mer oligonucleotides purchased from Operon (version 1.1), of approx 21,300 from human, onto glass slides.

A number of transcripts were altered on a constitutive basis in NOD2 expressing cells compared to NOD2 null cells irrespective of the presence of bacteria. Thus 138 transcripts were either increased more than 3-fold or reduced by at least 75% when RNA from these two types of cells was evaluated either before or after bacterial invasion. While a number of transcriptional changes appear to follow bacterial invasion irrespective of the presence of NOD2 and presumably are directed by non-NOD dependent pathway. However, distinctive alterations including both increased transcription of a small subset of genes and reduced expression of other genes appear to be dependent on NOD2 activation and are not seen in the absence of either NOD2 or bacterial invasion with Salmonella. In total, this amounts to 47 transcripts (from more than 12,500 evaluated) that are reproducibly increased by more than three fold or reduced by more than 80%.

The relevance of these transcripts is suggested by the absence of these observed changes in cells expressing the mutant NOD2 following Salmonella invasion. It is noteworthy that the “phenotype” of the mutant NOD2 expressing cells is largely due to the absence of the changes induced by the wild type NOD2 suggesting that most of the altered functional outcome reflect failure to activate responses.

### Example 8

### GRIM-19 Binds NOD2

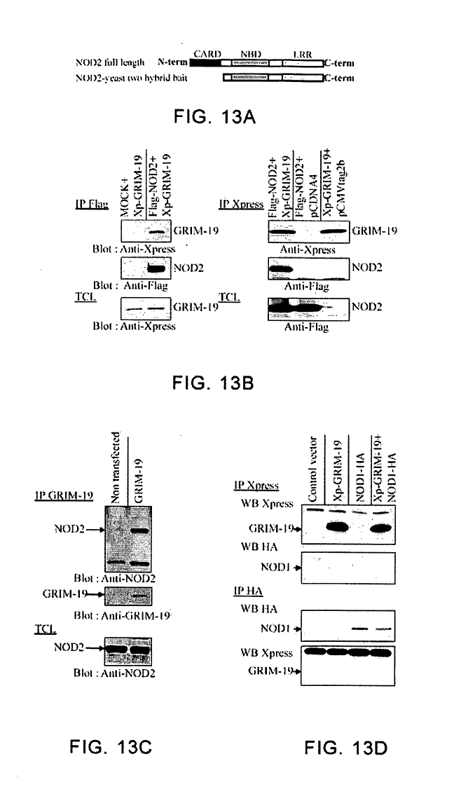

A yeast two-hybrid screen was performed to identify cellular proteins that interact with NOD2. A NOD2 protein containing an N-terminal deletion of the CARD15 domain was used as bait (FIG. 13A). A human bone marrow cDNA library expressing proteins fused to the AD transcriptional activation domain was screened. One positive clone was identified. This clone encodes the human GRIM-19 protein, a novel cell death-related gene (14). Co-expression of NOD2 and GRIM-19 in yeast survival assays in SD/-Ade/-His/-Leu/-Trp/X-Gal selective medium confirmed a strong interaction between these two proteins.

### Example 9

### Association of NOD2 and GRIM-19

To confirm the interaction of NOD2 and GRIM-19 in mammalian cells, COS7 or HEK293 cells were transfected with Flag-tagged NOD2 and Xpress-tagged GRIM-19. As shown in FIG. 13B, GRIM-19 was detected in anti-Flag immunoprecipitates from NOD2 co-transfectants, but not from cells co-transfected with the control plasmid. A reciprocal immunoprecipitation/blotting experiment with an anti-Xpress monoclonal antibody also showed NOD2 co-precipitating with GRIM-19 (FIG. 13B). To explore the physiological significance of the NOD2/GRIM-19 interaction, the endogenous interaction between NOD2 and GRIM-19 was investigated. The HM2559 rabbit antiserum against NOD2 was used (19). A rabbit antiserum against GRIM-19 was generated. The anti-GRIM-19 antibody specifically recognized Xpress-tagged GRIM-19 that was overexpressed in COS7 or HT29 cells. The rabbit antiserum against NOD2, HM2559, showed that endogenous NOD2 was highly expressed in HT29 cells, whereas COS7 and HEK293 cells expressed only low amount of endogenous NOD2. GRIM-19 was expressed in HT29 cells and associated with endogenous NOD2 as shown in an immunoprecipitation assay using anti-GRIM-19 antiserum (FIG. 13C). Similarly, immunoprecipitation assays with an anti-GRIM-19 antiserum showed that GRIM-19/NOD2 binding was increased in HT29 cells overexpressing NOD2 relative to untransfected HT29 cells. The interaction between GRIM-19 and CARD4/NOD1 was also examined. No association between NOD1 and GRIM-19 was identified by immunoprecipitation (FIG. 13D). These data suggest a specific functional link between GRIM-19 and NOD2.

### Example 10

### GRIM-19 and NOD2 Colocalize in Caco-2 and COS7 Cells

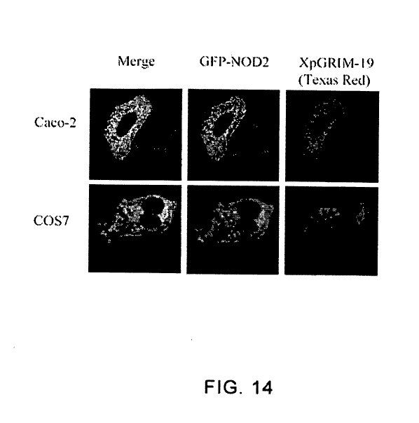

To determine the cellular compartment in which NOD2 and GRIM-9 interact, the subcellular localization of the two proteins was analyzed using immunofluorescence confocal microscopy. COS7 and Caco-2 cells were transfected with Xpress-GRIM-19 and GFP-NOD2 expression plasmids (FIG. 14). The NOD2 protein was observed throughout the cytoplasm and also near the plasma membrane. In COS7 and Caco-2 cells co-expressing GFP-NOD2 and Xpress-GRIM-19, GRIM-19 partially colocalized with NOD2 in intracellular vesicles, but not near the membrane (FIG. 14).

### Example 11

### GRIM-19 is Expressed in IBD Tissues and Intestinal Epithelial Cell Lines

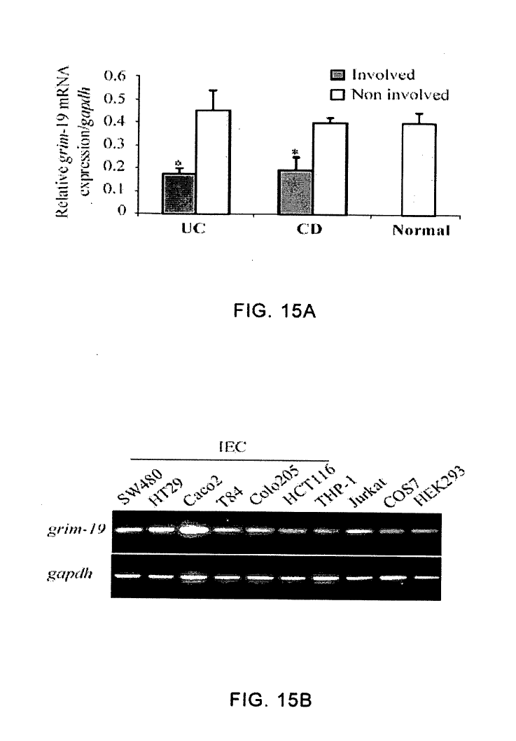

grim-19 mRNA expression was also analyzed in colonic biopsies from patients with Crohn's disease or ulcerative colitis. Biopsies were taken from both involved and noninvolved areas. GRIM-19 expression levels in these tissues was compared to expression levels in mucosal biopsies obtained from normal control patients without IBD. In the non-involved mucosa from IBD patients, grim-19 mRNA expression was comparable to that in control patients. In contrast, grim-19 mRNA expression was significantly decreased in involved areas from mucosa of both ulcerative colitis and Crohn's disease patients (FIG. 15A). Expression of grim-19 mRNA was also assessed by RT-PCR in several human intestinal epithelial cell lines, THP-1 macrophage cell line, and Jurkat cells. GRIM-19 was expressed in THP-1, Jurkat cell lines, and in all the IEC lines used in this study (FIG. 15B).

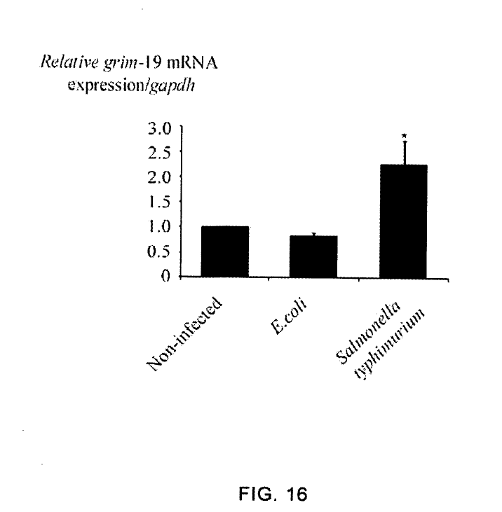

The effect of bacterial invasion on GRIM-19 expression was evaluated. Caco-2 cells were infected with invasive Salmonella typhimurium and non-pathogenic and non-invasive E. coli for two hours at a MOI 100. The fold variation of grim-19 mRNA levels was determined after non-invasive E. coli infection versus S. typhimurium infection in Caco-2 cells in comparison with gapdh mRNA levels. The size of PCR products was verified using 2% agarose gel electrophoresis The invasive ability of the S. typhimurium was verified using a gentamicin protection assay. S. typhimurium infection up-regulated grim-19 mRNA expression in Caco-2 cells (2.26-fold) while infection with a non-pathogenic and non-invasive E. coli had no effect on grim-19 mRNA expression (FIG. 16). Data shown are the mean ±SEM of four separate experiments (p<0.05).

### Example 12

### Functional Role of GRIM-19 in Caco-2 Cells

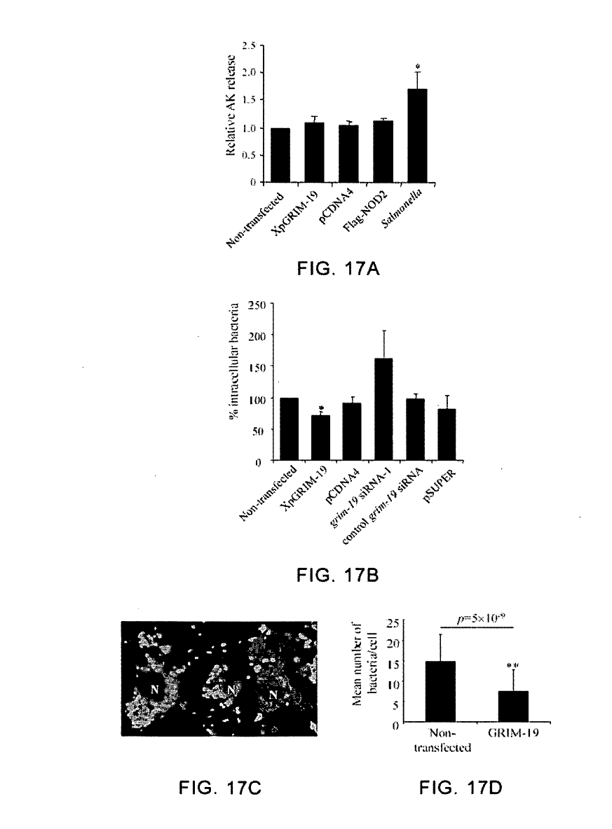

The effect of GRIM-19 on cell death was assayed using a non-destructive bioluminescence cytotoxicity assay on Caco-2 cells. Cells were transfected with Xpress-tagged GRIM-19 or Flag-tagged NOD2, or infected with S. typhimurium. Overexpression of GRIM-19 or NOD2 did not induce cell death in the transfected cells. Infection by S. typhimurium induced cell death in Caco-2 cells (FIG. 17A).

To determine whether GRIM-19 protein expression alters the functional outcome of bacterial survival, untransfected and transiently transfected Caco-2 cells were infected with S. typhimurium. S. typhimurium were incubated for two hours with the Caco-2 cell monolayer. The percentage of intracellular bacteria was significantly decreased (72.0%±5.4%) in Caco-2 cells expressing GRIM-19 when compared to the percentage of intracellular bacteria in untransfected control cells or in cells transfected with an empty control vector (FIG. 17B). Consistent with these findings, S. typhimurium invasion increased (162.0%±43.8%) in Caco-2 cells harboring a plasmid encoding the grim-19 siRNA-1 (FIG. 17B). This plasmid significantly decreased grim-19 mRNA levels. This effect was not observed in cells containing a grim-19 control siRNA (sequence 2), which did not affect grim-19 mRNA.

Immunostaining was performed on Caco-2 cells transfected with Xpress-tagged GRIM-19 and infected with S. typhimurium. As shown in FIG. 17C, virtually no bacteria were present in Caco-2 cells expressing GRIM-19, whereas numerous bacteria were observed in adjacent cells that not expressing GRIM-19 (FIG. 17C). Consistent with these results, the mean number of intracellular bacteria present in Caco-2 cells expressing GRIM-19, as shown by immunostaining examination by confocal microscopy, was significantly lower (7.5 bacteria/cell) (p<0.001) than the mean number of intracellular bacteria present in untransfected cells (14.8 bacteria/cells) (FIG. 17D). Thus, GRIM-19 protected host cells by preventing the intracellular survival of S. typhimurium.

### Example 13

### Retinoic Acid and IFN-α Exerts Anti-Bacterial Activity by Inducing GRIM-19 Expression

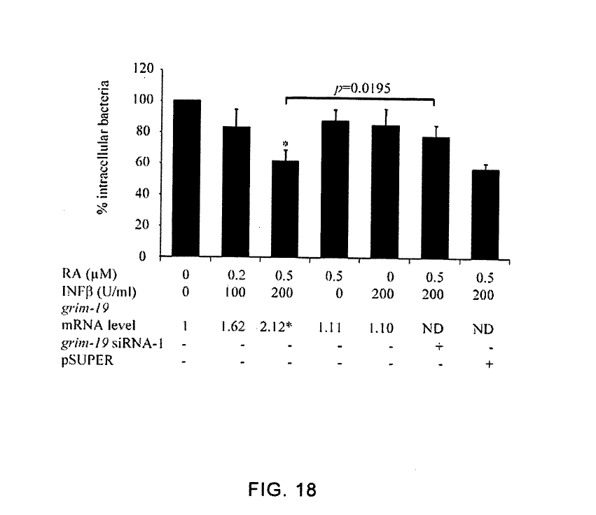

To investigate whether endogenous GRIM-19 exerts anti-microbial activity directly, endogenous GRIM-19 expression was induced by stimulating Caco-2 cells with a combination of RA and IFN-α. After stimulation with the combination of RA and IFN-α, real time RT-PCR showed that grim-19 mRNA expression was significantly increased (FIG. 18), whereas Caco-2 cells stimulated with either RA or IFN-α alone did not show increased GRIM-19 expression. Subsequently the ability of S. typhimurium to invade Caco-2 cells that were unstimulated or stimulated with retinoic acid (RA) or IFN-α or both was evaluated. After a two hour incubation period with the epithelial monolayer, the percentage of intracellular bacteria was significantly decreased in Caco-2 cells stimulated with both RA and IFN-α (FIG. 18), whereas the invasive ability of S. typhimurium remained the same in Caco-2 cells stimulated only with either RA or with IFN-α. Increased grim-19 expression correlated with a decrease in recovered viable S. typhimurium in bacterial invasion assays. Finally, grim-19 siRNA-1 restored the invasive ability of S. typhimurium in stimulated-Caco-2 cells in comparison with non-stimulated Caco-2 cells (FIG. 18), indicating that retinoic acid and IFN-α exert anti-bacterial activity by inducing endogenous GRIM-19 expression.

### Example 14

### GRIM-19 is Required for NOD2 Mediated NF-κB Activation

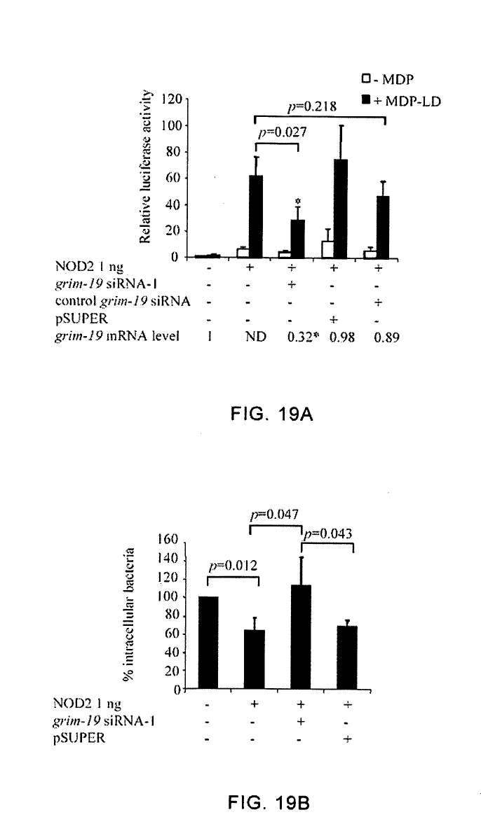

HEK293 cells transfected with 1 ng of NOD2 were stimulated with 1 μg of MDP-LD and transfected with grim-19 siRNA-1. Transfection with grim-19 siRNA-1 significantly decreased grim-19 mRNA level, and inhibited the MDP-LD driven-response to NOD2. NF-κB activation in HEK293 transfected with NOD2 and grim-19 siRNA-1 after MDP-LD stimulation was only 50% of that observed in HEK293 transfected only with NOD2, or with pSUPER control vector (FIG. 19A). Control grim-19 siRNA, which did not knockdown grim-19 mRNA levels, had no significant effect on NF-κB activation via NOD2 after MDP-LD stimulation.

The anti-bacterial activity of NOD2 was also dependent on the presence of GRIM-19. The invasive ability of S. typhimurium decreased in HEK293 cells overexpressing NOD2 compared to untransfected HEK293 cells (FIG. 19B). This effect was reversed in the presence of grim-19 siRNA-1 (FIG. 19B), indicating that anti-bacterial activity conferred by NOD2 correlates with NF-κB activation.

NOD2, but not the NOD2 mutant 3020insC was previously shown to be associated with Crohn's disease where it protects intestinal epithelial cells against Salmonella infection (19). In the present study, yeast two-hybrid screening identified GRIM-19 as an interacting protein with NOD2 in mammalian cells. GRIM-19, a gene associated with retinoid-IFN-induced mortality 19, is located on chromosome 19 and induces cell death in a number of tumor cell lines. GRIM-19 protein expression is induced by the combination of interferon-γ (IFN-1) and all-trans-retinoic acid (RA) (20, 21). The subcellular location of GRIM-19 action remains to be established. Originally GRIM-19 was observed in the nucleus (20) and more recently in both nucleus and cytoplasm (22). Its nuclear, but also cytoplasmic distribution and punctate staining patterns observed in cells prompted speculation that GRIM-19 might interact with various protein or protein complexes to regulate cellular responses (20, 21). GRIM-19 is also a subunit of the mitochondrial NADPH:ubiquinone oxidoreductase (respiratory complex I) (23) and co-localized with mitochondria in MCF-7 and COS-1 cells (24). Recently, GRIM-19 was detected in the native form in mitochondrial complex I. Homologous deletion of GRIM-19 in mice causes embryonic lethality at embryonic day 9.5 (25). In the present study, cytoplasmic colocalization of GRIM-19 and NOD2 was found. Furthermore, this interaction between GRIM-19 and NOD2 was NOD2-specific; no binding was observed with NOD1, another NOD protein family member.

In addition to NOD2, GRIM-19 binds proteins that play a crucial role in inflammatory bowel disease, including Stat3 and GW112. GRIM-19 binds Stat3 in various cell types, but did not bind other Stat proteins, such as Stat1 or Stat5a (24), and the interaction between GRIM-19 and Stat3 suppresses Stat3 activity. Stat3 has a critical role in the development and regulation of innate immunity, and deletion of Stat3 during hematopoiesis causes Crohn's disease-like pathogenesis and lethality in mice (26). GRIM-19 has also been reported to bind GW112, a protein expressed in various human normal and malignant tissues with higher expression in organs/tumors of the digestive system. GW112 plays an anti-apoptotic role that promotes tumor growth, and GW112 could be involved in the regulation of cellular apoptosis under inflammatory conditions in the digestive system (27).

Salmonella infection increased grim-19 mRNA in infected-Caco2 cells, whereas expression remained unchanged in Caco-2 cells infected with non-invasive E. coli. Epithelial cells of the human intestinal mucosa are the initial site of host invasion by bacterial enteric pathogens. Human colonic epithelial cells were shown to undergo apoptosis following infection with different invasive bacteria, such as enteroinvasive E. coli or Salmonella. Apoptosis in response to bacterial infection may eliminate infected and damaged epithelial cells and restore epithelial cell growth regulation and epithelial integrity (28). It has previously been shown that after invasion of intestinal macrophages, virulence proteins secreted by Salmonella specifically induce apoptotic cell death by activating the cysteine protease caspase-1 (29).

GRIM-19 has been shown to interact with multiple proteins such as mitochondrial NADH:ubiquinone oxidoreductase and to have several biological activities including cell growth, transcription and cell death (19). In the conditions of this study, GRIM-19 did not induce cell death. Expression of GRIM-19 in Caco-2 cells decreased the invasive ability of Salmonella, revealing its protective role in IEC. Given that the overall transfection efficiency in the Caco-2 cells was approximately 30-35%, the 28% reduction in CFU indicated that GRIM-19 was highly effective in controlling intracellular survival in cells expressing the transfected protein. Down-regulation of GRIM-19 expression using siRNA increased the invasive ability of S. typhimurium. Consistent with these findings, the invasive ability of S. typhimurium also decreased in Caco-2 cells expressing increased endogenous GRIM-19 induced by the combination of IFNγ/RA. In addition, siRNA against GRIM-19 restored invasive activity of S. typhimurium in Caco-2 cells stimulated with IFNγ/RA, confirming that endogenous GRIM-19 protects cells against bacteria. Expression of GRIM-19 was also decreased in inflammatory bowel disease affected areas obtained from patients having Crohn's disease or ulcerative colitis when compared to un-involved tissue. Without being tied to any particular theory, this decrease in GRIM-19 could be due to the loss of epithelium in the involved area, or to the down-regulation of GRIM-19 expression in inflamed areas in IBD patients. Given the protective role identified herein for GRIM-19, decreased GRIM-19 expression in involved colonic areas in IBD patients could enhance the ability of commensal and/or pathogenic bacteria to invade and/or survive in involved areas of the intestinal epithelium.

As described herein, GRIM-19 acts as a key component of the innate immune mucosal response by modulating NF-κB activation via NOD2. Decreased expression of GRIM-19 was achieved by treating HEK293 cells that overexpressed NOD2 with grim-19 siRNAs. This decreased NF-κB activation in these cells in response to MDP-LD. NFκB activation correlated with the decrease in number of viable intracellular S. typhimurium, indicating that NOD2 anti-bacterial activity was dependent on NF-κB activation.

In addition to its effects on NOD2 mediated NF-κB activation, it is likely that GRIM-19 has other functions that contribute to its effects on epithelial response to invasive bacteria. GRIM-19 is a subunit of the mitochondrial NADPH:ubiquinone oxidoreductase (23-25). The NADPH enzyme complex catalyzes the transfer of electrons from NADPH to molecular oxygen, generating reactive oxygen species (ROS), in particular superoxide anion. ROS operates on a variety of physiological processes, including host defense against pathogens (30). The best known O₂-producing enzyme is the phagocyte associated respiratory enzyme NADPH oxidase burst that plays a crucial role in a process of killing microorganisms (30). Helicobacter pylori LPS stimulated Toll-Like-receptor 4 signalling and activated the NADPH oxidase 1 (31,32). In addition, a yeast two hybrid screen led to the observation of a direct interaction of TLR4 with NADPH oxidase 4 which mediates LPS-induced ROS generation and NF-κB activation (33). Without being tied to any particular theory, it is possible that GRIM-19 increased the production of ROS in intestinal epithelial cells after bacterial invasion, thereby protecting the intestinal mucosa against pathogens. GRIM-19 could support mucosal defense by mediating NOD2 function in the recognition of bacterial pathogens and their elimination in intestinal epithelial cells.

The experiments described in the Examples above were carried out using the following methods and materials.

Cell Culture and Transfection

SW480, HT29, Caco-2, T84, Colo205, HCT116, HEK293, COS7, THP-1, and Jurkat cells were obtained from the American Type Culture Collection (Manassas, Va.). HEK293 and COS7 cells were cultured in Dulbecco's modified Eagle medium (Cellgro Mediatech Inc., Herndon, Va.) supplemented with 10% (vol/vol) heat-inactivated fetal calf serum (FCS, Atlanta Biologicals Inc., Norcross, Ga.). THP-1 and Jurkat cells were cultured in RPMI medium (Cellgro Mediatech Inc.) containing 10% heat-inactivated FCS. All the other IEC lines were cultured as described previously (19). Cells were transfected with a cationic lipid (LipofectAMINE 2000, Invitrogen, Carlsbad, Calif.) according to the manufacturer's protocols. For immunostaining experiments, cells were transfected using TransIT transfection reagents kit (Mirus corporation, Madison, Wis.) according to the manufacturer's instructions.

Yeast Two-Hybrid Screening

Yeast two-hybrid screening was performed using an enhanced GAL4 two-hybrid system, MATCHMAKER GAL4 TWO-HYBRID SYSTEM 3 (BD Biosciences Clontech, Palo Alto, Calif.), according to the manufacturer's instructions. Briefly, pGBKT7-NOD2 was generated by PCR methods from pCMVFlag-NOD2 vector (19). pGBKT7-NOD2 was transfected into the AH109 yeast strain. Expression of a Myc-tagged NOD2 protein in yeast extract was confirmed by Western blot analysis using anti-Myc monoclonal antibody (Covance, Richmond, Calif.) and affinity purified anti-NOD2 anti-sera (19). Screening was performed using a bone marrow pre-transformed library (BD Biosciences Clontech, Palo Alto, Calif.) according to the manufacturer's protocol. Co-transformants were selected in SD medium lacking Histidine, Leucine and Tryptophan. Yeast 13-galactosidase activity, expressed from the MEL1 gene in response to GAL4 activation, was determined in plates containing X-β-Gal (BD Biosciences Clontech).

Construction of Expression Plasmids

An Xpress-tagged GRIM-19 mammalian expression vector (pcDNA4/HisMAX-GRIM-19) was generated by PCR using cDNA from T84 cells. A Flag-tagged NOD2 mammalian expression vector (pCMVFlag-NOD2) was previously constructed (19) and GFP-tagged NOD2 mammalian expression vector (pEGFPC 1-NOD2) was generated by restriction methods from pCMVFlag-NOD2. The pCI CARD4/NOD1-HA expression vector was kindly provided by Dr. John Bertin (Millennium Pharmaceuticals Inc.). Two oligonucleotides, 19 residues in length (1-gtgtgggatactgcgagta and 2-atcgaggacttcgaggctc) and specific to the human grim-19 cDNA were selected for synthesis of siRNA (18). A vector for the expression of siRNAs, pSUPER vector, was purchased from Oligoengine (Seattle, Wash.). The reduction of endogenous GRIM-19 expression by siRNA was confirmed using RT-PCR.

Immunoprecipitation and Immunoblotting Experiments

Cells were grown on 6-well plates. Culture medium was removed and 300 μL1% Triton lysis buffer (1.25% sodium dodecyl sulfate, 2.5% glycerol, 62.5 mmol/L Tris/HCl, pH6.8, 5% 2-mercapto-ethanol) supplemented with protease-inhibitor cocktail (Complete Mini, Roche) was added to the cells. The cell lysate was spun at 12,000g for 10 minutes. The supernatant was reserved. Supernatant protein concentration was determined using the DC PROTEIN ASSAY KIT (Bio-Rad Laboratories, Hercules, Calif.). Two milligrams of cell lysate were immunoprecipitated with 2 μg of anti-Flag, anti-Xpress or anti-HA monoclonal antibodies and 100 μL Hiptrap protein A/G sepharose beads. After overnight incubation at 4° C., immunoprecipitated proteins were separated on 4% to 12% or on 4% to 20% Tris-Glycine gel (Invitrogen). Proteins were blotted onto polyvinylidene difluoride (PVDF) membranes and stained for Flag-NOD2 using anti-Flag monoclonal antibody (Sigma-Aldrich), for Xpress-GRIM-19 using anti-Xpress monoclonal antibody (Invitrogen) and for NOD1-HA using anti-HA monoclonal antibody (Roche). For endogenous binding, 500 μg of total protein from HT29 cells were subjected to immunoprecipitation/blotting as described above using rabbit anti-NOD2 antiserum HM2559 (19) and affinity purified rabbit antiserum against human GRIM-19 produced by Affinity Bioreagents (Project#A203001, Immunizing peptide: IMKDVPDWKVGESVF).

Confocal Microscopy

Caco-2 cells were grown on sterile permanox coverslips for twenty-four hours, then transfected with pcDNA4/HisMAX-GRIM-19 and pCMVFlag-NOD2. After forty-eight hours, the cells were washed twice with ice-cold PBS, fixed 20 minutes with cold methanol at −20° C., and washed three times with ice-cold PBS. Cells were saturated for 30 minutes with PBS containing 5% donkey serum. Cells were then incubated for two hours with primary antibody (mouse monoclonal anti-Xpress antibody and/or rabbit polyclonal anti-Flag antibody (Sigma)). Immunostaining was performed with Texas-Red-conjugated anti-mouse IgG or with FITC-conjugated anti-rabbit IgG (Vector Laboratories) secondary antibodies. S. typhimurium were detected with an anti-Salmonella rabbit antibody conjugated to fluorescein (Biodesign International, Saco, Me.). Coverslips were mounted in Vectashield (Vector Laboratories) and examined with a confocal laser scanning microscope.

Bacterial Invasion Assays

Invasion assays were performed with Salmonella enterica serovar Typhimurium or Escherichia coli TOP10 (Invitrogen). Cell monolayers were seeded in 24-well tissue culture plate with 10⁵ cells/well and incubated for 20 hours. Monolayers were then infected in 1 ml of cell culture medium without antibiotic and with heat-inactivated FCS at a multiplicity of infection (MOI) of 10 bacteria per epithelial cell. After a two hour incubation period at 37° C., the monolayers were washed two times with PBS. Fresh cell culture medium containing 100 μg/ml of gentamicin (Sigma) was then added for one hour to kill any extracellular bacteria. The epithelial cells were then lysed with 1% Triton X-100 in deionized water. Samples of cell lysates were diluted and plated onto Luria-Bertani agar plates to determine the number of colony forming units (cfu), which corresponds to the number of intracellular bacteria.

Reverse-Transcription Polymerase Chain Reaction

Total RNA of IEC lines were extracted using Trizol (INVITROGEN) according to the manufacturer's instructions. For reverse transcription, 2 μg of total RNA was transcribed with RT PCR reagents provided in the SUPERSCRIPT FIRST-STRAND SYNTHESIS SYSTEM (INVITROGEN). Real time RT-PCR was performed in an ABI Prism 7000 Sequence Detector using SYBR Green JumpStart™ detection system. Briefly, 50 ng of the reversed transcribed cDNA were used for each PCR reaction with 200 nM of forward and reverse primers. Primers used for PCR had the following sequences: Forward 5-accggaagtgtgggatactg-3, Reverse 5-gctcacggttccacttcatt-3 (GRIM-19, 194 bp); Forward 5-tcatctctgccccctctgct-3, Feverse 5-cgacgcctgcttcaccacct-3 (glyceraldehyde-3-phosphate dehydrogenase [GAPDH], 440 bp). The following PCR program was used: 50° C. for 2 minutes, then 95° C. for 10 minutes followed by 40 cycles of 95° C. for 15 seconds, 60° C. for 15 seconds and 72° C. for 15 seconds. The threshold cycle (C_{T}) values were obtained for the reactions reflecting quantity of the template in the sample. GRIM-19 Delta C_{T} (ΔC_{T}) was calculated by subtracting the GAPDH C_{T} value from the GRIM-19 C_{T} value and thus, represented the relative quantity of the target molecule after normalizing with the internal standard GAPDH. The GRIM-19 ΔC_{T} values of Caco-2 cells infected with Salmonella, or transfected with pcDNA4/HisMAX-GRIM 19 were expressed as the percentage of GRIM-19 ΔC_{T} values of control Caco-2 cells.

PCR products were sequenced using a ABI 3700 PRISM (Perkin Elmer, Boston, Mass.) automated sequencer. Sequences were analyzed using NCBI BLAST software.

Cytotoxicity Assays

To evaluate the effect of overexpressing GRIM-19 and NOD2 in Caco-2 cells, the release of adenylate kinase from damage cells was measured using a ToxiLight non-destructive cytotoxicity assay according to the manufacturer's instructions (Cambrex Bio Science, Rockland, Me.). As a positive control, Caco-2 cells were also infected with Salmonella typhimurium at a MOI=50 for two hours.

NF-κB Activation Assays

HEK293 cells were transfected overnight with 1 ng of NOD2, 10 ng of grim-19 siRNA plus 1 ng of pIV luciferase reporter plasmid and renilla plasmid. At the same time, 1 μg of MDP-LD (Sigma) was added. After twenty-four hours, luciferase activity was measured using the Dual-Luciferase Reporter Assay System (Promega, Madison, Wis.) according to the manufacturer's instructions and normalized relative to renilla activity.

Statistical Analysis

The Student's t-test was used to analyze the statistical significance of differences between data sets for invasion levels, NF-κB levels, and mRNA levels. All experiments were repeated at least three times. A P-value equal or less than 0.05 was considered to be statistically significant.

### REFERENCES

All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.
1. Athman et al., (2004) Curr Opin Microbiol 7, 25-32
2. Ogura et al., (2001) Nature 411, 603-606
3. Hugot et al., (2001) Nature 411, 599-603
4. Ogura et al., (2001) J Biol Chem 276, 4812-4818
5. Chen et al., (2004) J Biol Chem 279, 25876-25882
6. Opitz et al., (2004) J Biol Chem 279, 36426-36432
7. Hisamatsu et al., (2003) Gastroenterology 124, 993-1000
8. Chin et al., (2002)Nature 416, 190-194
9. Kobayashi et al., (2002)Nature 416, 194-199
10. Bertin et al., (1999) J Biol Chem 274, 12955-12958
11. Inohara et al., (1999) J Biol Chem 274, 14560-14567
12. Inohara et al., (2003) J Biol Chem 278, 5509-5512
13. Girardin et al., (2003) J Biol Chem 278, 8869-8872
14. Barnich et al., Mol Microbiol 2003; 48:781-94.
15. Betis et al., Infect Immun 2003; 71:1774-83.
16. Darfeuille-Michaud et al., Gastroenterology 1998; 115:1405-13.
17. Glasser et al., Infect Immun 2001; 69:5529-37.
18. Brummelkamp, T. R., Bernards, R., and Agami, R. (2002) Science 296, 550-553
19. Hisamatsu et al., (2003) Gastroenterology 124, 993-1000
20. Angell et al., (2000) J Biol Chem 275, 33416-33426
21. Chidambaram et al., (2000) J Interferon Cytokine Res 20, 661-665
22. Hu et al., (2002) J Interferon Cytokine Res 22, 1017-1026
23. Feamley et al., (2001) J Biol Chem 276, 38345-38348
24. Lufei et al., (2003) Embo J 22, 1325-1335
25. Huang et al., (2004) Mol Cell Biol 24, 8447-8456
26. Welte et al., (2003) Proc Natl Acad Sci USA 100, 1879-1884
27. Zhang et al., (2004) Cancer Res 64, 2474-2481
28. Kim et al., (1998) J Clin Invest 102, 1815-1823
29. Jarvelainen et al., (2003) Trends Cell Biol 13, 204-209
30. Babior, (2004) Curr Opin Immunol 16, 42-47
31. Teshima et al., (1999) FEBS Lett 452, 243-246
32. Kawahara et al., (2001) Infect Immun 69, 4382-4389
33. Park et al., (2004) J Immunol 173, 3589-3593

A number of embodiments of the invention have been described. Nevertheless, it will be understood that various modifications may be made without departing from the spirit and scope of the invention. Accordingly, other embodiments are within the scope of the following claims.

1.-4. (canceled)

5. A method for identifying a compound that decreases an intestinal inflammation, the method comprising the steps of:
(a) contacting a cell expressing a GRIM-19 polypeptide with a candidate compound; and
(b) detecting an increase in the amount of GRIM-19 polypeptide in the cell contacted with the candidate compound relative to an amount of a reference polypeptide,
wherein an increase in the amount of GRIM-19 polypeptide identifies the candidate compound as a compound that decreases an intestinal inflammation.

6. A method for identifying a compound that decreases an intestinal inflammation, the method comprising the steps of:
(a) contacting a cell expressing a GRIM-19 polypeptide with a candidate compound; and
(b) comparing the biological activity of the GRIM-19 polypeptide in the cell contacted with the candidate compound with the biological activity of the GRIM-19 polypeptide in a control cell,
wherein an increase in the biological activity of the GRIM-19 polypeptide identifies the candidate compound as a compound that decreases an intestinal inflammation.

7. The method of claim 6, wherein the biological activity is monitored with an enzymatic assay.

8. The method of claim 7, wherein the enzymatic assay detects nicotinamide adenine dinucleotide phosphate dehydrogenase activity.

9. The method of claim 6, wherein the biological activity is monitored with an NF-κB activation assay.

10. The method of claim 6, wherein the biological activity is monitored with a bacterial invasion assay.

11. The method of claim 6, wherein the biological activity is monitored with an immunological assay.

12. The method of claim 11, wherein the immunological assay detects GRIM-19 binding to NOD2.

13. A method of identifying a compound that decreases an intestinal inflammation, the method comprising the steps of:
a) contacting a cell comprising a GRIM-19 promoter operably linked to a detectable reporter gene with a candidate compound; and
b) comparing the amount of reporter gene expression in the cell contacted with the candidate compound with a control cell not contacted with the candidate compound,
wherein an increase in the amount of the reporter gene expression identifies the candidate compound as a compound that decreases an intestinal inflammation.

14. The method of claim 5, wherein the cell is in vitro.

15. The method of claim 5, wherein the cell is in vivo.

16. The method of claim 14, wherein the cell is an intestinal epithelial cell.

17. A method for identifying a compound that decreases an intestinal inflammation, the method comprising the steps of:
(a) contacting a GRIM-19 polypeptide with a candidate compound;
(b) detecting binding of the GRIM-19 polypeptide with the candidate compound; and
(c) monitoring the biological activity of the Grim 19 polypeptide,
wherein an increase in the biological activity of the GRIM-19 polypeptide thereby identifies the candidate compound as a compound that decreases an intestinal inflammation.

18. The method of claim 17, wherein the binding is detected in a cell.

19. The method of claim 5, wherein the compound alters a host response to a microbe.

20. The method of claim 19, wherein the microbe is a bacteria.

21. The method of claim 5, wherein the candidate compound is a small molecule, a nucleic acid molecule, or a polypeptide.

22. The method of claim 5, wherein the method is a high throughput screening method.

23. The method of claim 5, wherein the candidate compound is an antibiotic.

24. The method of claim 23, wherein the antibiotic is useful for treating an infection or inflammation that occurs anywhere in the body.

25. (canceled)

26. A method for diagnosing a subject having, or having a propensity to develop, an intestinal inflammation, the method comprising detecting an alteration in: the sequence of a GRIM-19 nucleic acid molecule relative to a wild-type sequence of a GRIM-19 nucleic acid molecule, the expression of a GRIM-19 nucleic acid molecule or polypeptide relative to the wild-type level of expression of the GRIM-19 nucleic acid molecule or polypeptide, or the biological activity of a GRIM-19 polypeptide relative to the wild-type level of activity.

27. (canceled)

28. (canceled)

29. The method of claim 26, wherein the intestinal inflammation is an inflammatory bowel disease.

30. The method of claim 29, wherein the inflammatory bowel disease is Crohn's disease or ulcerative colitis.

31. A method for ameliorating an intestinal inflammation in a subject, the method comprising contacting the subject with one or more compounds that increase: GRIM-19 nucleic acid or polypeptide expression or GRIM-19 activity, thereby ameliorating the intestinal inflammation in the subject.

32. (canceled)

33. The method of claim 31, wherein one of the compounds is an interferon or retinoic acid.

34. The method of claim 31, wherein the compounds are an interferon and retinoic acid.

35. The method of claim 31, wherein the intestinal inflammation is associated with an inflammatory bowel disease.

36. The method of claim 35, wherein the inflammatory bowel disease is Crohn's disease or ulcerative colitis.

37. A method for reducing a pathogen infection in a subject, the method comprising contacting the subject with one or more compounds that increase: GRIM-19 nucleic acid or polypeptide expression or GRIM-19 activity, thereby reducing the pathogen infection in the subject.

38. (canceled)

39. A method for inactivating a pathogen in an epithelial cell, the method comprising providing the cell with a GRIM-19 nucleic acid molecule or polypeptide, or an activator thereof.

40. The method of claim 37, wherein the pathogen is a bacteria.

41. The method of claim 40, wherein the bacteria is E. coli or S. typhimuriam.

42. The method of claim 40, wherein the method inhibits the growth or survival of the bacteria.

43-45. (canceled)

46. A method of inhibiting microbial growth in a cell, the method comprising providing an effective amount of a biocide comprising a GRIM-19 polypeptide or a nucleic acid molecule or fragment thereof to a cell containing the microbe.

47-70. (canceled)
